(19) United States
(12) Patent Application Publication
(10) Pub. No.: US 2023/0287032 A1
(43) Pub. Date: Sep. 14, 2023

(54) SYNTHESIS OF FLUORINATED NUCLEOTIDES

(71) Applicants: Cheol Keun CHUNG, Rahway, NJ (US); Zhijian LIU, Kendall Park, NJ (US); Peter E. MALIGRES, Rahway, NJ (US); Edna MAO, Plainsboro, NJ (US); Jennifer V. OBLIGACION, Rahway, NJ (US); Eric M. PHILLIPS, Boston, MA (US); Michael PIRNOT, Rahway, NJ (US); Marc POIRIER, Rahway, NJ (US); Zhiguo Jake SONG, Freemont, CA (US); Tao WANG, Rahway, NJ (US); Merck Sharp & Dohme LLC, Rahway, NJ (US)

(72) Inventors: Cheol Keun Chung, Westfield, NJ (US); Zhijian Liu, Kendall Park, NJ (US); Peter E. Maligres, Fanwood, NJ (US); Edna Mao, Plainsboro, NJ (US); Jennifer V. Obligacion, Metuchen, NJ (US); Eric M. Phillips, Jersey City, NJ (US); Michael Pirnot, Milford, NJ (US); Marc Poirier, Stewartsville, NJ (US); Zhiguo Jake Song, Fremont, CA (US); Tao Wang, Cranford, NJ (US)

(73) Assignee: Merck Sharp & Dohme LLC, Rahway, NJ (US)

(21) Appl. No.: 18/040,679

(22) PCT Filed: Aug. 11, 2021
(86) PCT No.: PCT/US2021/045465

§ 371 Date: Feb. 6, 2023

Related U.S. Application Data
(60) Provisional application No. 63/065,732, filed on Aug. 14, 2020.

(51) Int. Cl. C07H 19/16

(52) U.S. Cl. CPC C07H 19/16

(57) ABSTRACT

The present invention relates to efficient processes useful in the preparation of fluorinated nucleosides, such as (O—{[(2R,3R,4S,5R)-5-(6-amino-9H-purin-9-yl)-4-fluoro-3-hydroxyoxolan-2-yl]methyl}O,O-dihydrogen phosphorothioate, also known as 2′-(S)-fluoro-thio-adenosine monophosphate or 2′-F-thio-AMP. Such fluorinated nucleosides may be useful as a biologically active compound and or as an intermediate for the synthesis of more complex biologically active compounds. The present invention also encompasses intermediates useful in the disclosed synthetic processes and the methods of their preparation.

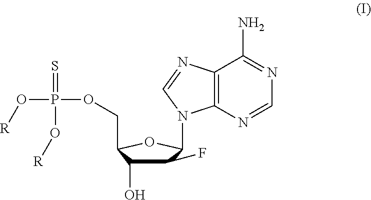

### FIELD OF THE INVENTION

The present invention relates to efficient synthetic processes useful in the preparation of fluorinated nucleotides, such as (O—{[(2R,3R,4S,5R)-5-(6-amino-9H-purin-9-yl)-4-fluoro-3-hydroxyoxolan-2-yl]methyl}O,O-dihydrogen phosphorothioate, also known as 2′-(S)-fluoro-thio-adenosine monophosphate or 2′-F-thio-AMP. Such fluorinated nucleotides may be useful as biologically active compounds and or as intermediates for the synthesis of more complex biologically active compounds. The present invention also encompasses intermediates useful in the disclosed synthetic processes and the methods of their preparation.

### BACKGROUND OF THE INVENTION

The synthesis of complex nucleotides and nucleosides continues to challenge the synthetic community, notwithstanding many years of attempts motivated by their medicinal importance. J. J. Fox, et al., Chapter 10: Antiviral Activities of 2′-Fluorinated Arabinosyl-Pyrimidine Nucleosides, in Fluorinated Carbohydrates—Chemical and Biochemical Aspects, ACS Symposium Series, Vol. 374, 176-190 (1988). Nucleosides containing fluorine stereocenters can greatly enhance the desired biological activity of such nucleosides. However, the introduction of such stereocenters adds another level of difficulty beyond the already challenging synthesis. See, e.g., X.-L. Qui et al., Recent Advances In The Synthesis Of Fluorinated Nucleosides, 66 TETRAHEDRON 789-843 (2010). Early approaches suffered from poor yields and required manipulation of protecting groups at several steps.

While these syntheses were promising as improved methods for the preparation of fluorinated nucleobase analogs, they employ lengthy synthetic sequences, among other disadvantages. Thus, there remains a need for improved syntheses of 2′-fluorinated nucleosides, and in particular 2′-F-thio-AMP nucleotide.

### SUMMARY OF THE INVENTION

The present disclosure relates to processes useful in the synthesis of 2′-fluorinated nucleotides, particularly 2′-F-thio-AMP. The present disclosure also encompasses chemical processes that afford intermediates useful in the production of 2′-F-thio-AMP. The chemical processes of the present disclosure afford advantages over previously known procedures and include a more efficient route to 2′-fluorinated nucleotides by using a synthetic design that relies on stereo-controlled electrophilic fluorination and glycosylation. The processes rely on a new class of organocatalysts that reduce the formation of dimeric and polymeric impurities during glycal formation. Subsequent fluorination at the 2′ position followed by nucleobase addition produces 2′-fluorinated nucleosides, such as 2′-F-adenosine, that demonstrate good control of stereoselectivity for both fluorination and nucleobase addition steps. These processes, described in further detail herein, provide a shorter synthetic route. Additionally, these processes use inexpensive raw materials, avoid the use of corrosive and hazardous aminosulfurane-based fluorinating reagents such as diethylaminosulfur trifluoride (DAST) and bis(2-methoxyethyl) aminosulfur trifluoride (BAST) to install fluorine, use organocatalysts to improve efficiency in two key steps, and proceed exclusively through stable, crystalline intermediates.

In addition, the disclosure relates to crystalline forms of 2′-F-thio-AMP that have been identified herein.

Other embodiments, aspects and features of the present disclosure are either further described in or will be apparent from the ensuing description, examples and appended claims.

### DETAILED DESCRIPTION OF THE INVENTION

### Definitions

Terms used herein have their ordinary meaning, which is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name, and an ambiguity exists between the structure and the name, the structure predominates. These definitions apply regardless of whether a term is used by itself or in combination with other terms, unless otherwise indicated. Hence, the definition of “alkyl” applies to “alkyl” as well as the “alkyl” portions of “hydroxyalkyl,” “haloalkyl,” “—O-alkyl,” etc.

As used herein, and throughout this disclosure, the following terms, unless otherwise indicated, shall be understood to have the following meanings:

The term “alkyl,” as used herein, refers to an aliphatic hydrocarbon group having one of its hydrogen atoms replaced with a bond having the specified number of carbon atoms. In different embodiments, an alkyl group contains from 1 to 6 carbon atoms (C₁-C₆ alkyl) or from 1 to 3 carbon atoms (C₁-C₃ alkyl). Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. In one embodiment, an alkyl group is linear. In another embodiment, an alkyl group is branched.

The terms “halogen” and “halo,” as used herein, means —F (fluorine), —Cl (chlorine), —Br (bromine) or —I (iodine).

The term “haloalkyl,” as used herein, refers to an alkyl group as defined above, wherein one or more of the alkyl group's hydrogen atoms has been replaced with a halogen. In one embodiment, a haloalkyl group has from 1 to 6 carbon atoms. In another embodiment, a haloalkyl group has from 1 to 3 carbon atoms. In another embodiment, a haloalkyl group is substituted with from 1 to 3 halogen atoms. Non-limiting examples of haloalkyl groups include —CH₂F, —CHF₂, and —CF₃. The term “C₁-C₄ haloalkyl” refers to a haloalkyl group having from 1 to 4 carbon atoms.

The term “alkoxy” as used herein, refers to an —O-alkyl group, wherein an alkyl group is as defined above. Non-limiting examples of alkoxy groups include methoxy, ethoxy, n-propoxy, isopropoxy, n-butoxy and tert-butoxy. An alkoxy group is bonded via its oxygen atom to the rest of the molecule.

The term “aryl,” as used herein, refers to an aromatic monocyclic or multicyclic ring system comprising from about 6 to about 14 carbon atoms. In one embodiment, an aryl group contains from about 6 to 10 carbon atoms (C₆-C₁₀ aryl). In another embodiment an aryl group is phenyl. Non-limiting examples of aryl groups include phenyl and naphthyl.

When a functional group in a compound is termed “protected,” the group is in modified form to preclude undesired side reactions at the protected site when the compound is subjected to a reaction. The term “PG”, as used herein, refers to a protecting group. Those skilled in the art will readily envisage protecting groups (PG) suitable for use in compounds and processes according to the disclosure. Suitable protecting groups will be recognized by those of ordinary skill in the art as well as by reference to standard textbooks such as, for example, T. W. Greene et al., Protective Groups in Organic Synthesis (1991), Wiley, New York. Protecting groups suitable for use herein include acid-labile protecting groups. Non-limiting examples of PG suitable for use herein include —S(O)₂R⁸, —C(O)OR⁸, —C(O)R⁸, —CH₂OCH₂CH₂SiR⁸, and —CH₂R⁸, wherein R⁸ is selected from the group consisting of —C₁₋₈ alkyl (straight or branched), —C₃₋₈ cycloalkyl, —CH₂(aryl), and —CH(aryl)₂, wherein each aryl is independently phenyl or naphthyl and each said aryl is optionally independently unsubstituted or substituted with one or more (e.g., 1, 2, or 3) groups independently is selected from —OCH₃, Cl, Br, and I.

The term “substituted” means that one or more hydrogens on the atoms of the designated moiety are replaced with a selection from the indicated group, provided that the atoms' normal valencies under the existing circumstances are not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

When any substituent or variable occurs more than one time in any compound, its definition on each occurrence is independent of its definition at every other occurrence, unless otherwise indicated. For example, description of radicals that include the expression “—N(C₁-C₃ alkyl)₂” means —N(CH₃)(CH₂CH₃), —N(CH₃)(CH₂CH₂CH₃), and —N(CH₂CH₃)(CH₂CH₂CH₃), as well as —N(CH₃)₂, —N(CH₂CH₃)₂, and —N(CH₂CH₂CH₃)₂.

It should also be noted that any carbon or heteroatom with unsatisfied valences in the text, schemes, examples and Tables herein is assumed to have sufficient hydrogen atom(s) to satisfy the valences. Any one or more of these hydrogen atoms can be deuterium.

One or more compounds herein may exist in unsolvated as well as solvated forms with pharmaceutically acceptable solvents, such as water, ethanol, and the like, and this disclosure is intended to embrace both solvated and unsolvated forms. “Solvate” means a physical association of a compound with one or more solvent molecules. This physical association involves varying degrees of ionic and covalent bonding, including hydrogen bonding. In certain instances of this aspect, the solvate will be capable of isolation, for example when one or more solvent molecules are incorporated in the crystal lattice of a crystalline solid. “Solvate” encompasses both solution-phase and isolatable solvates. Non-limiting examples of suitable solvates include ethanolates, methanolates, and the like. “Hydrate” is a solvate in which the solvent molecule is H₂O.

Compounds herein may contain one or more stereogenic centers and can thus occur as racemates, racemic mixtures, single enantiomers, diastereomeric mixtures and individual diastereomers. Additional asymmetric centers may be present depending upon the nature of the various substituents on the molecule. Each such asymmetric center will independently produce two optical isomers, and all possible optical isomers and diastereomers in mixtures and as pure or partially purified compounds are included within the disclosure. Any formulas, structures, or names of compounds described herein that do not specify a particular stereochemistry are meant to encompass any and all existing isomers as described above and mixtures thereof in any proportion. When stereochemistry is specified, the disclosure is meant to encompass that particular isomer in pure form or as part of a mixture with other isomers in any proportion.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Enantiomers can also be separated by use of chiral HPLC column.

All stereoisomers (for example, geometric isomers, optical isomers, and the like) of disclosed compounds (including those of the salts and solvates of compounds as well as the salts, solvates and esters of prodrugs), such as those that may exist due to asymmetric carbons on various substituents, including enantiomeric forms (which may exist even in the absence of asymmetric carbons), rotameric forms, atropisomers, and diastereomeric forms, are contemplated within the scope of this disclosure. Individual stereoisomers of compounds may, for example, be substantially free of other isomers, or may be admixed, for example, as racemates or with all other, or other selected, stereoisomers. The chiral centers can have the or R configuration as defined by the IUPAC 1974 Recommendations.

Compounds can form salts that are also within the scope of this disclosure. Reference to a compound herein is understood to include reference to salts thereof, unless otherwise indicated. The term “salt(s),” as employed herein, denotes acidic salts formed with inorganic and/or organic acids, as well as basic salts formed with inorganic and/or organic bases. In addition, when a compound contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds may be formed, for example, by reacting a compound with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids that are generally considered suitable for the formation of pharmaceutically useful salts from basic pharmaceutical compounds are discussed, for example, by P. Stahl et al., Camille G. (eds.) Handbook of Pharmaceutical Salts: Properties, Selection and Use (2002) Zurich: Wiley-VCH; S. Berge et al., J. Pharm. Sci. (1977) 66(1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C.). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, tert-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

The present disclosure further includes compounds and synthetic intermediates in all their isolated forms. For example, the above-identified compounds are intended to encompass all forms of the compounds such as, any solvates, hydrates, stereoisomers, and tautomers thereof.

The present disclosure also embraces isotopically-labelled compounds that are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl and ¹²³I, respectively.

Certain isotopically-labelled compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Isotopic substitution at a site where epimerization occurs may slow or reduce the epimerization process and thereby retain the more active or efficacious form of the compound for a longer period of time. Isotopically labeled compounds, in particular those containing isotopes with longer half lives (T_{1/2}>1 day), can generally be prepared by following procedures analogous to those disclosed in the Schemes and/or in the Examples herein below, by substituting an appropriate isotopically labeled reagent for a non-isotopically labeled reagent.

Those skilled in the art will recognize that chiral compounds, and in particular sugars, can be drawn in a number of different ways that are equivalent. Those skilled in the art will further recognize that the identity and regiochemical position of the substituents on ribose can vary widely and that the same principles of stereochemical equivalence apply regardless of substituent. Non-limiting examples of such equivalence include those exemplified below.

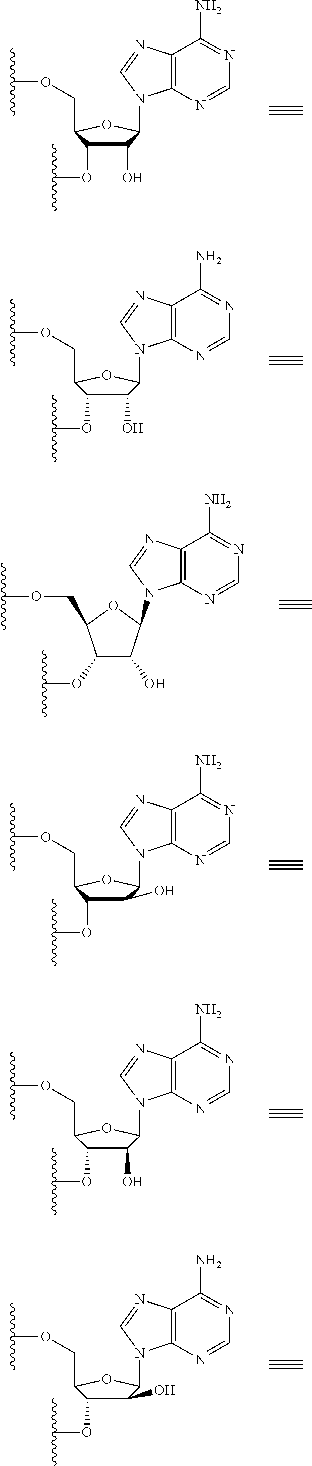

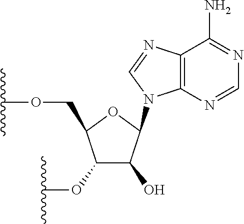 [SMILES: *OC[C@H]1O[C@@H](n2cnc3c(N)ncnc32)[C@@H](O)[C@@H]1O*]

Similarly, those skilled in the art will recognize that certain compounds, and in particular compounds containing certain heteroatoms and double or triple bonds, can be tautomers, structural isomers that readily interconvert. Thus, tautomeric compounds can be drawn in a number of different ways that are equivalent. Non-limiting examples of such tautomers include those exemplified below.

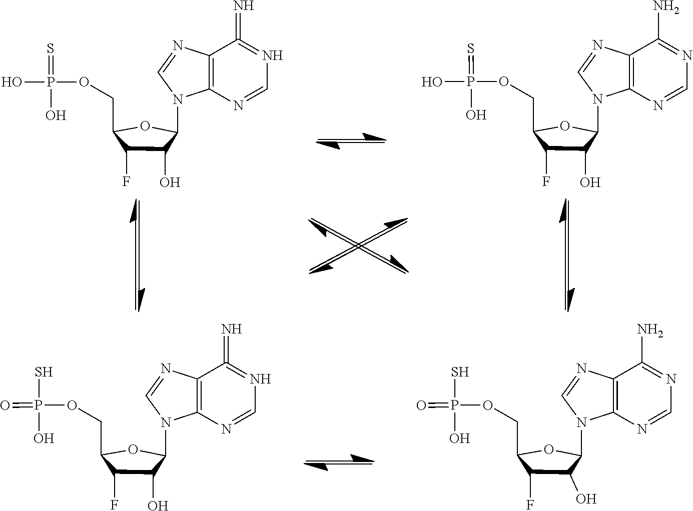

### ABBREVIATIONS

2,4,6-collidine 2,4,6-Trimethylpyridine
2,6-lutidine 2,6-Dimethylpyridine
2-Me-THF 2-Methyltetrahydrofuran
Ac Acetyl
ACN, MeCN Acetonitrile
AcOH, HOAc Acetic acid
aq Aqueous
BAST bis(2-Methoxyethyl)aminosulfur trifluoride
Bn Benzyl
BSA Bistrimethylsilyl acetamide
BSTFA bistrimethylsilyl trifluoroacetamide, also referred to as trimethylsilyl 2,2,2-trifluoro-N-(trimethylsilyl)acetimidate
Bu Butyl
Bz Benzoyl
CPME Cyclopentylmethyl ether
DABCO 1,3-Diazabicyclo[2.2.2]octane
DAST Diethylaminosulfur trifluoride
DBSI N,N-Dibenzenesulfonimide
DBU 1,8-Diazabicyclo[5.4.0]undec-7-ene
DCA Dichloroacetic acid
DCM, CH₂C₁₂ Dichloromethane
DI water Deionized water
DMAc Dimethylacetamide
DME, Glyme Dimethoxyethane
DMF N,N-Dimethylformamide
DMPU N,N′-Dimethylpropyleneurea
eq Equivalents
EtOAc Ethyl acetate
g Grams
h Hour
H₂O Water
HDMS Hexamethyldisilazane
HFIP Hexafluoro-2-propanol
Hunig's Base N,N-Diisopropylethylamine
IPA Isopropyl alcohol
IPAc Isopropyl acetate
M Molar, moles per liter
mg Milligrams
MIBK Methyl isobutyl ketone
min Minute(s)
mL or ml Milliliters
mM Millimole per liter
mmol Millimoles
Ms Methanesulfonyl
MTBE Methyl tert-butyl ether, methyl tertiary butyl ether
NFSI N-fluorobenzenesulfonimide
NMI 1-Methylimidazole
PG Protecting group
PIV or Piv pivalate or 2,2-Dimethylpropanoate
PTPI N,N-bis(diphenylthiophosphoryl)amide
PSePI N,N-bis(diphenylselenophosphoryl)amide
Py Pyridine
RPM, rpm Revolutions per minute
RT Room temperature, approximately 25° C.
TBS tert-Butyldimethylsilyl
Tetraglyme Tetraethylene glycol dimethyl ether
TFA Trifluoroacetic acid
TFE 2,2,2-Trifluoroethanon
THF Tetrahydrofuran
Trityl Triphenylmethyl
Ts para-Toluenesulfonyl (tosyl)
UPLC Ultra Performance Liquid Chromatography
Vol. Volumes
XG Xyloguanosine
2′-F-thio-AMP (O—{[(2R,3R,4S,5R)-5-(6-amino-9H-purin-9-yl)-4-fluoro-3-hydroxyoxolan-2-yl]methyl}O,O-dihydrogen phosphorothioate, also known as 2′-fluoro-thio-adenosine monophosphate

The present disclosure provides a process for preparing compounds of Formula (I) and pharmaceutically acceptable salts, hydrates, and solvates thereof:

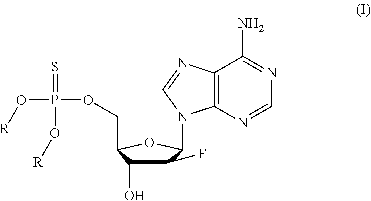

wherein each R is independently is selected from the group consisting of H, Na, and K. In specific embodiments, the disclosure provides a process for preparing a compound of Formula (Ia), 2′-F-thio-AMP, and pharmaceutically acceptable salts, hydrates, and solvates thereof:

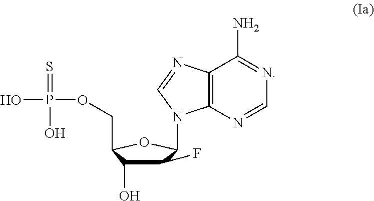

A first embodiment comprises reacting a compound of Formula (I-1) with a thiophosphorylating agent in the presence of at least one Catalyst A and at least one Base A in the presence of at least one Solvent A, to form an intermediate compound of Formula (I-1¹) and then quenching with Quenching Reagent A to form a compound of Formula (I).

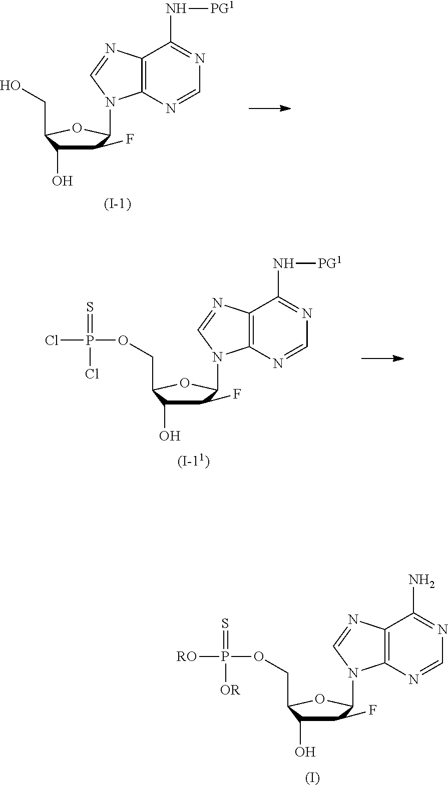

In compounds of Formula (I-1), each R is as defined above, and PG¹ is selected from the group consisting of H, isobutyryl, pivaloyl, benzoyl, acetyl, octanoyl, and 2-ethyl-hexanoyl. In particular aspects of this embodiment, PG¹ is pivaloyl (PIV or Piv).

In a first aspect of the first embodiment, the thiophosphorylating agent is selected from the group consisting of PSCl₂OK and PSCl₃. In instances of this aspect of this first aspect of the first embodiment, the thiophosphorylating agent is PSCl₃. In specific instances of this aspect, the thiophosphorylating agent is provided in an amount in a range of from about 0.5 to about 5.0 equivalents with respect to the amount of the compound of Formula (I-1), such as an 15 amount in a range of from about 0.75 to about 3.5 equivalents, an amount in a range of from about 1.0 to about 2.5 equivalents, or an amount in a range of from about 1.5 to about 2.0 equivalents.

In a second aspect of the first embodiment, the at least one Catalyst A is selected from the group consisting of N-methyl morpholine, N-methyl imidazole, N-methyl-benzimidazole, quinine,

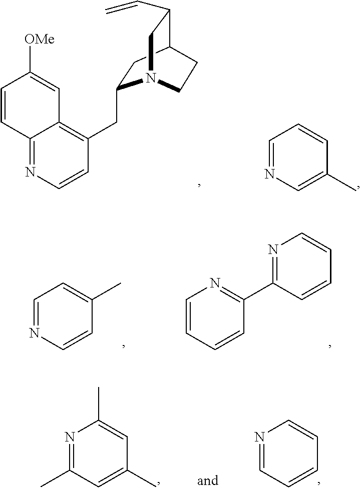

and mixtures thereof. In instances of this aspect, the at least one Catalyst A is selected from

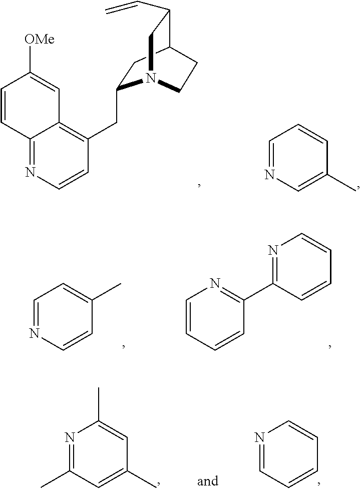

and mixtures thereof. In specific instances of this aspect, the Catalyst A is

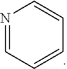

In specific instances of this aspect, the at least one Catalyst A is provided in an amount in a range of from about 0.01 to about 5.0 equivalents with respect to the amount of the compound of Formula (I-1), such as an amount in a range of from about 0.1 to about 1.0 equivalents, an amount in a range of from about 0.15 to about 0.4 equivalents, or an amount of about 0.25 equivalents.

In a third aspect of the first embodiment, the at least one Base A is selected from the group consisting of 2,6-lutidine, pyridine, 4-picoline, pyridine, 2-picoline, quinoline, 2-F-pyridine, 2,4-lutidine, 2-methyl-pyridine, 2,4,6-trimethylpyridine, 2,3,5-trimethylpyridine, 3-methoxy-pyridine, 4-methyl-pyridine, quinuclidine, Hunig's base, triethylamine, 3-methyl-pyridine, and 2,6-di-tert-butyl-4-methyl pyridine, N-methyl morpholine, and mixtures thereof. In specific instances of this aspect, the at least one Base A is selected from the group consisting of 2,6-lutidine, 2,4,6-trimethylpyridine, 2,3,5-trimethylpyridine, 2,4-dimethylpyridine, and pyridine, and mixtures thereof. In still more specific instances of this aspect, the at least one Base A is 2,6-lutidine. In specific instances of this aspect, the at least one Base A is provided in an amount in a range of from about 0.5 to about 5.0 equivalents with respect to the amount of the compound of Formula (I-1), such as an amount in a range of from about 1.0 to about 3 equivalents, or an amount of about 1.5 equivalents.

In a fourth aspect of the first embodiment, the at least one Solvent A is selected from the group consisting of THF, MeCN, acetone, DMPU, HFIP, TFE, glyme, DME, DMAc, propylene carbonate, tetraglyme, trimethyl phosphate, triethyl phosphate, 2-Me-THF, EtOAc, and MIBK, and mixtures thereof. In instances of this aspect, the at least one Solvent A is selected from the group consisting of tetraglyme, MeCN, trimethyl phosphate, and triethyl phosphate, and mixtures thereof. In specific instances of this aspect, the at least one Solvent A is selected from triethyl phosphate and tetraglyme, and mixtures thereof. In particular specific instances of this aspect, the at least one Solvent A is triethyl phosphate. In specific instances of this aspect, the at least one Solvent A is provided in an amount in a range of from about 3 to about 50 volumes with respect to the amount of the compound of Formula (I-1), such as an amount in a range of from about 3 to about 20 volumes, or an amount of about 5 volumes.

In a fifth aspect of the first embodiment, the reacting to form the compound of Formula (I-1¹) is conducted at a temperature in a range of from about −20° C. to about 30° C., such as at a temperature in a range of from about −10° C. to about 10° C., or about −5° C.

In a sixth aspect of the first embodiment, the reaction forming the compound of Formula (I-1¹) is quenched from at least one Quenching Reagent A is selected from the group consisting of water, water in combination with pyridine, or water in combination with one or more additives, where said additives are independently is selected from guanidine-HCl, phenol, sodium dodecyl sulfate, thiourea, lithium acetate, magnesium chloride, and urea, and mixtures thereof. In specific instances of this aspect, the at least one Quenching Reagent A is water. In specific instances of this aspect, the at least one Quenching Reagent A is provided in an amount in a range of from about 0.5 to about 20 volumes with respect to the amount of the compound of Formula (I-1), such as an amount in a range of from about 0.5 to about 5 volumes, or an amount of about 2 volumes.

In a seventh aspect of the first embodiment, the reaction forming the compound of Formula (I) is conducted by heating the reaction at a temperature in a range of from about 20° C. to about 100° C., such as at a temperature in a range of from about 30° C. to about 60° C., or about 50° C. In specific instances of this aspect, the reaction is aged for a duration in a range of from about 30 mins to 20 h, such as a duration in a range of from about 1 h to 5 h, or a duration of about 3 h.

In an eighth aspect of the first embodiment, the process further comprises isolating the compound of Formula (I) by crystallization from at least one Solvent B is selected from the group of water, methanol, ethanol, isopropanol, and mixtures thereof. In instances of this aspect, the Solvent B is water. In specific instances of this aspect, the at least one Solvent B is provided in an amount in a range of from about 0.5 to about 20 volumes with respect to the amount of the compound of Formula (I-1), such as an amount in a range of from about 2 to about 15 volumes, or an amount of about 9 volumes.

In a second embodiment, the disclosure provides a process for the preparation of the compound of Formula (I-1) from a compound of Formula (I-2). In particular, the process comprises preparing a compound of Formula (I-2¹) from a compound of Formula (I-2), followed by preparing a compound of Formula (I-2²) from a compound for Formula (I-2¹), followed by preparing a compound of Formula (I-1) from a compound for Formula (I-2²):

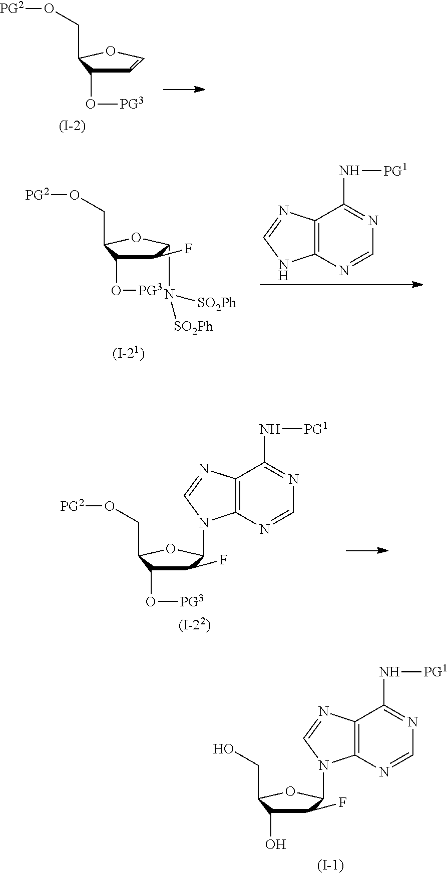

wherein PG¹ is as described above; PG² is selected from the group consisting of acyl, alkyl, and silyl; and PG³ is selected from the group consisting of acyl, alkyl, and silyl. In aspects of this embodiment, PG² is selected from the group consisting of isobutyryl, pivaloyl, trityl, tert-butyldiphenylsilyl, tert-butyldimethylsilyl, triisopropylsilyl, and trimethylsilyl; and PG³ is selected from the group consisting of isobutyryl, pivaloyl, trityl, tert-butyldiphenylsilyl, tert-butyldimethylsilyl, triisopropylsilyl, and trimethylsilyl. In particular aspects of the second embodiment, PG² is trimethylsilyl or tert-butyldimethylsilyl; and PG³ is trimethylsilyl or tert-butyldimethylsilyl. In more particular aspects of the second embodiment, PG² and PG³ are trimethylsilyl.

In a first aspect of the second embodiment, the disclosure provides a process for the preparation of the intermediate compound of Formula (I-2¹) by reacting a compound of Formula (I-2) with at least one Fluorinating Agent A in the presence of at least one Base B, at least one Silylating Reagent A, and at least one Solvent C to provide a compound of Formula (I-2¹):

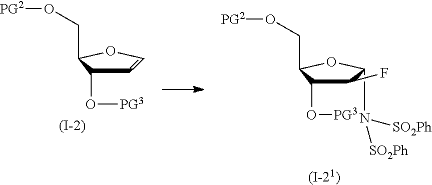

In a first instance of the first aspect of the second embodiment, the at least one Fluorinating Agent A is selected from the group consisting of N-fluorobenzenesulfonimide, 1-chloromethyl-4-fluoro-1,4-diazoniabicyclo[2.2.2] octane bis(tetrafluoroborate) (also known as F-TEDA; commercially available from SigmaAldrich as SELECTFLUOR™), 1-fluoro-4-methyl-1,4-diazoniabicyclo[2.2.2] octanebis(tetrafluoroborate) (also known as N-fluoro-N′-methyl-triethylenediamine bis(tetrafluoroborate); commercially available from SigmaAldrich as SELECTFLUOR II™) N-fluoropyridinium triflate, and N-fluoropyridinium tetrafluoroborate, and mixtures thereof. In specific occurrences of this instance, the at least one Fluorinating Agent A is N-fluorobenzenesulfonimide. In additional occurrences of this instance, the Fluorinating Agent A is provided in an amount in a range of from about 1.10 to about 2.0 equivalents with respect to the amount of the compound of Formula (I-2), or an amount of about 1.10 equivalents.

In a second instance of the first aspect of the second embodiment, the at least one Base B is selected from the group consisting of pyridine, 2,6-lutidine, triethylamine, N-methylmorpholine, 2,4,6-collidine, potassium carbonate, dibasic potassium phosphate, tribasic potassium phosphate, and sodium bicarbonate, and mixtures thereof. In occurrences of this instance, the at least one Base B is 2,6-lutidine. In additional occurrences of this instance, the at least one Base B is provided in an amount in a range of from about 0.1 to about 0.9 equivalents with respect to the amount of the compound of Formula (I-2), or an amount of about 0.5 equivalents.

In a third instance of the first aspect of the second embodiment, the at least one Silylating Reagent A is selected from the group consisting of 1,3-bis(trimethylsilyl)urea, 3-(trimethylsilyl)-oxazolidin-2-one, hexamethyldisilazane (HDMS), bistrimethylsilyl acetamide (BSA), bistrimethylsilyl trifluoroacetamide (BSTFA, also referred to as trimethylsilyl 2,2,2-trifluoro-N-(trimethylsilyl)acetimidate), and N-trimethylsilylimidazole, and mixtures thereof. In occurrences of this instance, the at least one Silylating Reagent A is bistrimethylsilyl trifluoroacetamide. In additional occurrences of this instance, the Silylating Reagent A is provided in an amount in a range of from about 0.05 to about 2.0 equivalents with respect to the amount of the compound of Formula (I-1), or an amount of about 0.05 equivalents.

In a fourth instance of the first aspect of the second embodiment, the at least one Solvent C is selected from the group consisting of ethyl acetate, dioxane, dimethoxyethane, tetrahydrofuran, 2-methyltetrahydrofuran, cyclopentyl methylether, methyl tert-butyl ether, 1,2-dichloroethane, acetonitrile, isopropyl acetate, diethylcarbonate, and toluene, and mixtures thereof. In occurrences of this instance, the at least one Solvent C is toluene. In additional occurrences of this instance, the Solvent C is provided in an amount in a range of from about 5 to about 20 volumes with respect to the amount of the compound of Formula (I-2), or an amount of about 5 volumes.

In a second aspect of the second embodiment, the disclosure provides a process for the preparation of the intermediate compound of Formula (I-2²) by reacting a compound of Formula (I-2¹) with a protected adenine in the presence of at least one Base C, and at least one Solvent D to provide a compound of Formula (I-2¹):

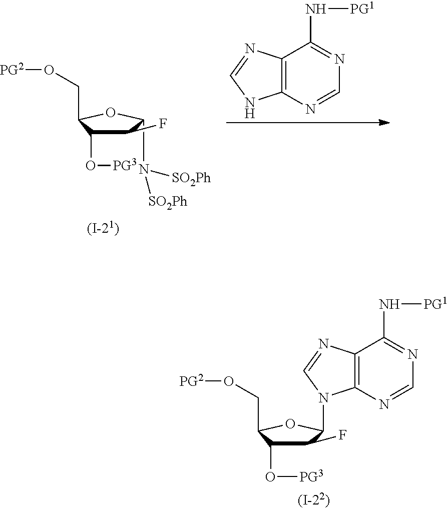

wherein PG¹, PG², and PG³ are as described above.

In a first instance of the second aspect of the second embodiment, the at least one Base C is selected from the group consisting of N,N-diisopropylethylamine, 2,6-lutidine, 2,2,6,6-tetramethylpiperidine, potassium acetate, potassium tert-butoxide, potassium carbonate, tribasic potassium phosphate, N-methylmorpholine, potassium bicarbonate, sodium bicarbonate, sodium carbonate, cesium carbonate, DBU, 2,4,6-collidine, DABCO, N-methylimidazole, 2,6-ditert-butyl-4-methyl pyridine, potassium tert-butoxide, and potassium hexamethyldisilazide, and mixtures thereof. In occurrences of this instance, the at least one Base C is 2,6-lutidine. In additional occurrences of this instance, the at least one Base C is provided in an amount in a range of from about 0.75 to about 3.0 equivalents with respect to the amount of the compound of Formula (I-2¹), or an amount of about 1.2 equivalents.

In a second instance of the second aspect of the second embodiment, the at least one Solvent D is selected from the group consisting of toluene, tetrahydrofuran, acetonitrile, dimethylformamide, acetone, dimethylacetamide, cyclopentyl methyl ether, dimethoxyethane, diethylcarbonate, 2-methyl tetrahydrofuran, isopropyl acetate, and ethyl acetate, and mixtures thereof. In particular occurrences of this instance, the at least one Solvent D is ethyl acetate. In additional occurrences of this instance, the at least one Solvent D is provided in an amount in a range of from about 5 to about 30 volumes with respect to the amount of the compound of Formula (I-2¹), or an amount of about 20 volumes.

In a third aspect of the second embodiment, the disclosure provides a process for preparing the compound of Formula (I-1) by reacting a compound of Formula (I-2¹) with at least one Acid A in the presence of at least one Solvent E to provide a compound of Formula (I-1):

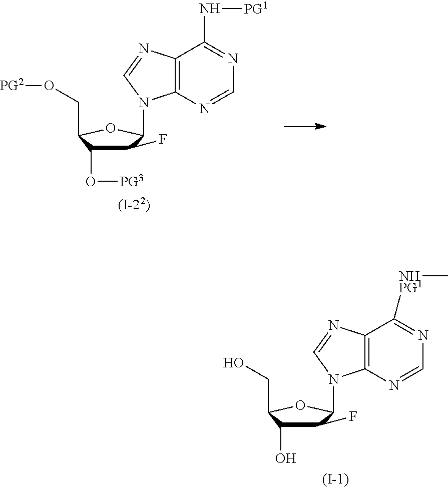

In a first instance of this third aspect of the second embodiment, the at least one Acid A is selected from the group consisting of TFA, HCl, H₂SO₄, MsOH, TsOH, and mixtures thereof. In occurrences of this instance, the at least one Acid A is TFA. In specific instances of this aspect, the at least one Acid A is provided in an amount in a range of from about 0.1 to about 8.0 equivalents with respect to the amount of the compound of Formula (I-2²), or an amount of about 0.1 equivalents.

In a second instance of the third aspect of the second embodiment, the at least one Solvent E is selected from the group consisting of isopropanol, methanol, water, ethyl acetate, and ethanol, and mixtures thereof. In particular occurrences of this instance, the at least one Solvent E is ethanol. In additional occurrences of this instance, the at least one Solvent E is provided in an amount in a range of from about 0.5 to about 5.0 volumes with respect to the amount of the compound of Formula (I-2¹), or an amount of about 0.5 volumes.

In a third embodiment, the disclosure provides a process for the preparation of the compound of Formula (I-2) by reacting a compound of Formula (I-3) with PG²-Cl and PG³-Cl in the presence of at least one Base D, at least one Silylating Reagent B, at least one Catalyst B, and at least one Solvent F to provide a compound of Formula (I-2):

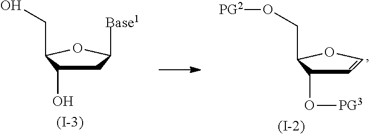

wherein Basel is selected from the group consisting of thymine, uracil, cytosine, N-acetylcytosine, guanine, and hypoxanthine, and PG² and PG³ are as described above. In aspects of the third embodiment, Basel is thymine.

In a first aspect of the third embodiment, compound (I-3) is selected from the group consisting of 2′-deoxynucleosides. In instances of this aspect, the compound (I-3) is selected from the group consisting of thymidine, 2′-deoxyluridine, 2′-deoxycytidine, 2′-deoxyguanosine, and 2′-deoxyinosine. In particular instances of this aspect, the compound (I-3) is thymidine.

In a second aspect of the third embodiment, PG²-X is selected from the group consisting of acyl halides, alkyl halides, and silyl halides; and PG³-X is selected from the group consisting of acyl halides, alkyl halides, and silyl halides. In instances of this aspect, PG²-X is selected from the group consisting of isobutyryl chloride, pivaloyl chloride, trityl chloride, tert-butyldiphenylsilyl chloride, tert-butyldimethylsilyl chloride, triisopropylsilyl chloride, and trimethylsilyl chloride, and PG³-X is selected from the group consisting of trityl chloride, tert-butyldiphenylsilyl chloride, tert-butyldimethylsilyl chloride, triisopropylsilyl chloride, and trimethylsilyl chloride. In specific instances of this aspect, PG²-X is tert-butyldimethylsilyl chloride, and PG³-X is tert-butyldimethylsilyl chloride. In additional specific instances of this aspect, PG²-X is trimethylsilyl chloride, and PG³-X is trimethylsilyl chloride. In more specific instances of this aspect, PG²-X and PG³-X are provided in a combined amount in a range of from about 2.0 to about 3.0 equivalents with respect to the amount of the compound of Formula (I-3), or an amount of about 2.1 equivalents.

In aspects of the third embodiment, the preparation of the compound of Formula (I-2) from a compound of Formula (I-3) is accomplished by one step that also includes the protection of hydroxyl groups with PG² and PG³, such as when PG² and PG³ are each trimethylsilyl, or may be accomplished in a series of steps. When the preparation of the compound of Formula (I-2) from a compound of Formula (I-3) is accomplished by a series of steps, one step includes protection of the hydroxyl groups with PG² and PG³ by reacting a compound of Formula (I-3) with PG²-Cl and PG³-Cl in the presence of at least one Base D, and in a subsequent step, said reacting is performed in the presence of at least one Silylating Reagent B, at least one Catalyst B, at least one Base E, and at least one Solvent F. The at least one Base E is selected from the group consisting of amines, and mixtures thereof. In instances of this aspect, the at least one Base E is selected from the group consisting of 2,6-lutidine, 2,4,6-collidine, Hunig's base, triethylamine, and mixtures thereof. In specific instances of this aspect, the at least one Base E is 2,6-lutidine. In specific instances of this aspect, the at least one Base E is provided in an amount in a range of from about 0 to about 1 equivalent with respect to the amount of the compound of Formula (I-3), or an amount of about 0.02 equivalents.

In a third aspect of the third embodiment, the at least one Base D is selected from the group consisting of amines, and mixtures thereof. In instances of this aspect, the at least one Base D is selected from the group consisting of Hunig's Base, imidazole, pyridine, NMI, 2,6-lutidine, 2,4,6-collidine, DBU, DABCO, tetramethylguanidine, triethylamine, diisopropylethylamine, and mixtures thereof. In specific instances of this aspect, the at least one Base D is imidazole. In specific instances of this aspect, the at least one Base D is provided in an amount in a range of from about 1.0 to about 5.0 equivalents with respect to the amount of the compound of Formula (I-3), or an amount of about 3.0 equivalents.

In a fourth aspect of the third embodiment, the at least one Silylating Reagent B is selected from the group consisting of 1,3-bis(trimethylsilyl)urea, 3-(trimethylsilyl)-oxazolidin-2-one, hexamethyldisilazane (HDMS), bistrimethylsilyl acetamide (BSA), bistrimethylsilyl trifluoroacetamide (BSTFA), and N-trimethylsilylimidazole, and mixtures thereof. In specific instances of this aspect, the at least one Silylating Reagent B is bistrimethylsilyl acetamide. In specific instances of this aspect, the at least one Silylating Reagent B is provided in an amount in a range of from about 2.0 to about 5.0 equivalents with respect to the amount of the compound of Formula (I-3), or an amount of about 4.5 equivalents.

In a fifth aspect of the third embodiment, the at least one Catalyst B is selected from the group consisting of Bronsted acid catalysts, and mixtures thereof. In instances of this aspect, the at least one Catalyst B is selected from the group consisting of mineral acids, sulfonic acids, sulfonimides, N-acylsulfonamides, electron poor sulfonamides, dialkyl-phosphine sulfides, dialkyl- phosphine selenides, diaryl-phosphine sulfides, diaryl-phosphine selenides, thio- and seleno-phosphinic and thio- and seleno-phosphoric acids, bis(thiophosphoryl)amides and bis(selenophosphoryl)amides, and bis(thiophosphoryl)amides and bis(selenophosphoryl)amides, and mixtures thereof. In specific instances of this aspect, the at least one Catalyst B is selected from the group consisting of sulfuric acid, methanesulfonic acid, N,N-bistriflimide, 1,2-phenyldisulfonimide, N,N-dibenzenesulfonimide (DBSI), N,N-bis(4-methoxybenzenesulfonyl) amide, N-(4-chlorobenzenesulfonyl)-N-methanesulfonylamide, N-benzenesulfonyl-benzamide, N,N-bis(methanesulfonyl)amide, saccharin, thiosaccharin, 6-nitrosaccharin, 6-chlorosaccharin, 5-fluorosaccharin, perfluorobenzenesulfonamide, diphenyldithiophosphinic acid, diethyldithiophosphoric acid, N,N-bis(diphenylthiophosphoryl) amide (PTPI), and N,N-bis(diphenylselenophosphoryl)amide (PSePI), and mixtures thereof. In more specific instances of this aspect, the at least one Catalyst B is selected from the group consisting of DBSI, PTPI, and PSePI, and mixtures thereof. In other specific instances of this aspect, the at least one Catalyst B is provided in an amount in a range of from about 0.001 to about 0.1 equivalents with respect to the amount of the compound of Formula (I-3), or an amount of about 0.01 equivalents.

In a sixth aspect of the third embodiment, said reacting is performed in the presence of at least one Solvent F, which is selected from the group consisting of hydrocarbons, halocarbons, ethers, and silanes, and mixtures thereof. In instances of this aspect, the at least one Solvent F is selected from the group consisting of dichloromethane, dichloroethane, hexane, heptane, cyclohexane, CPME, toluene, trifluorotoluene, hexamethyldisiloxane, hexamethyldisilazane (HMDS), and mixtures thereof In specific instances of this aspect, the at least one Solvent F is selected from the group consisting of heptane, toluene, and mixtures thereof. In specific instances of this aspect, the at least one Solvent F is provided in an amount in a range of from about 0 to about 20 volumes with respect to the amount of the compound of Formula (I-3), or an amount of about 10 volumes.

In a seventh aspect of the third embodiment, the compound of Formula (I-2) is purified by adding the reaction mixture to an alcohol, such as methanol, ethanol, and 2-propanol, or a mixture of alcohols, to induce selective alcoholysis and precipitation of by-products. In instances of this aspect, the at least one alcohol is provided in an amount in a range of from about 4 to about 20 molar equivalents with respect to the amount of the compound of Formula (I-3). In a more specific instance of this aspect, the at least one alcohol is 2-propanol.

In a fourth embodiment, the disclosure provides a process for the preparation of compounds of Formula (I), or a pharmaceutically acceptable salt, hydrate, or solvate thereof:

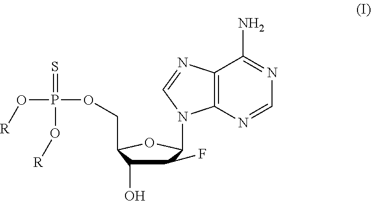

wherein each R is independently is selected from the group consisting of H, Na, and K, the process comprising
i) reacting a compound of Formula (I-3) with PG²-X and PG³-X in the presence of at least one Base D, at least one Silylating Reagent B, at least one Catalyst B, and at least one Solvent F to provide a compound of Formula (I-2):

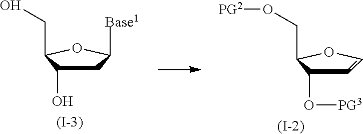

wherein
a) Base¹ is selected from the group consisting of thymine, uracil, cytosine, N-acetylcytosine, guanine, and hypoxanthine;
b) PG²-X is selected from the group consisting of acyl halides, alkyl halides, and silyl halides;
c) PG³-X is selected from the group consisting of acyl halides, alkyl halides, and silyl halides;
d) PG² is selected from the group consisting of acyl halides, alkyl halides, and silyl halides;
e) PG³ is selected from the group consisting of acyl halides, alkyl halides, and silyl halides;
f) the at least one Base D is selected from the group consisting of amines, and mixtures thereof;
g) the at least one Silylating Reagent B is selected from the group consisting of 1,3-bis(trimethylsilyl)urea, 3-(trimethylsilyl)-oxazolidin-2-one, hexamethyldisilazane, bistrimethylsilyl acetamide, bistrimethylsilyl trifluoroacetamide, and N-trimethylsilylimidazole, and mixtures thereof;
h) the at least one Catalyst B is selected from the group consisting of Bronsted acid catalysts, and mixtures thereof;
i) the at least one Solvent F is selected from the group consisting of hydrocarbons, halocarbons, ethers, and silanes, and mixtures thereof; and
j) the compound of Formula (I-2) may be optionally purified by adding the reaction mixture to an alcohol or mixture of alcohols to induce a selective alcoholysis and precipitation of by-products;
ii) reacting a compound of Formula (1-2) with at least one Fluorinating Agent A in the presence of at least one Base B, at least one Silylating Reagent A, and at least one Solvent C to provide a compound of Formula (I-2¹):

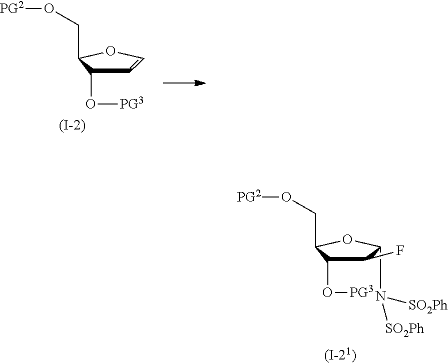

wherein
a) the at least one Fluorinating Agent A is selected from the group consisting of N-fluorobenzenesulfonimide, 1-chloromethyl-4-fluoro-1,4-diazoniabicyclo[2.2.2] octane bis(tetrafluoroborate), 1-fluoro-4-methyl-1,4-diazoniabicyclo[2.2.2] octanebis (tetrafluoroborate), N-fluoropyridinium triflate, and N-fluoropyridinium tetrafluoroborate, and mixtures thereof;
b) the at least one Base B is selected from the group consisting of pyridine, 2,6-lutidine, triethylamine, N-methylmorpholine, 2,4,6-collidine, potassium carbonate, dibasic potassium phosphate, tribasic potassium phosphate, and sodium bicarbonate, and mixtures thereof;
c) the at least one Silylating Reagent A is selected from the group consisting of 1,3-bis(trimethylsilyl)urea, 3-(trimethylsilyl)-oxazolidin-2-one, hexamethyldisilazane, bistrimethylsilyl acetamide, bistrimethylsilyl trifluoroacetamide, and N-trimethylsilylimidazole, and mixtures thereof; and
d) the at least one Solvent C is selected from the group consisting of ethyl acetate, dioxane, dimethoxyethane, tetrahydrofuran, 2-methyltetrahydrofuran, cyclopentyl methylether, methyl tert-butyl ether, 1,2-dichloroethane, acetonitrile, isopropyl acetate, diethylcarbonate, and toluene, and mixtures thereof;
iii) reacting a compound of Formula (I-2¹) with a protected adenine in the presence of at least one Base C, and at least one Solvent D to provide a compound of Formula (I-2²):

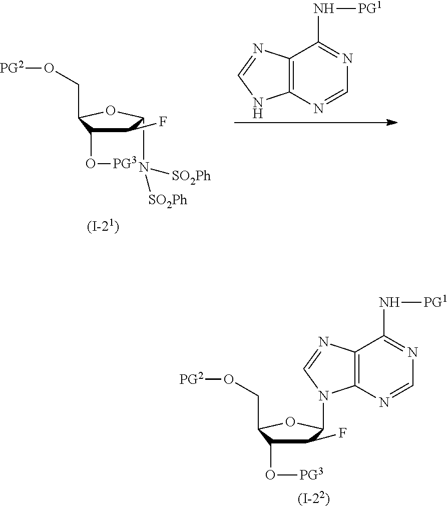

wherein
a) PG¹ is selected from the group consisting of H, isobutyryl, pivaloyl, benzoyl, acetyl, octanoyl, 2-ethyl-hexanoyl;
b) the at least one Base C is selected from the group consisting of N,N-diisopropylethylamine, 2,6-lutidine, 2,2,6,6-tetramethylpiperidine, potassium acetate, potassium tert-butoxide, potassium carbonate, tribasic potassium phosphate, N-methylmorpholine, potassium bicarbonate, sodium bicarbonate, sodium carbonate, cesium carbonate, DBU, 2,4,6-collidine, DABCO, N-methylimidazole, 2,6-ditert-butyl-4-methyl pyridine, potassium tert-butoxide, and potassium hexamethyldisilazide, and mixtures thereof; and
c) the at least one Solvent D is selected from the group consisting of toluene, tetrahydrofuran, acetonitrile, dimethylformamide, acetone, dimethylacetamide, cyclopentyl methyl ether, dimethoxyethane, diethylcarbonate, 2-methyl tetrahydrofuran, isopropyl acetate, and ethyl acetate, and mixtures thereof;
iv) reacting a compound of Formula (I-2²) with at least one Acid A in the presence of at least one Solvent E to provide a compound of Formula (I-1):

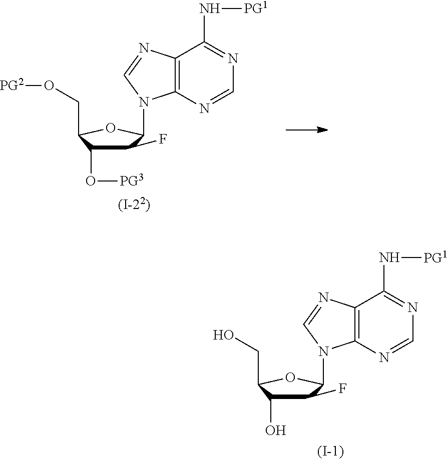

wherein
a) the at least one Acid A is selected from the group consisting of TFA, HCl, H₂SO₄, MsOH, TsOH, and mixtures thereof; and
b) the at least one Solvent E is selected from the group consisting of isopropanol, methanol, water, ethyl acetate, and ethanol, and mixtures thereof; and
v) reacting the compound of Formula (I-1) with a thiophosphorylating agent in the presence of at least one Catalyst A and at least one Base A in the presence of at least one Solvent A, to form an intermediate compound of Formula (I-1¹) and then quenching with Quenching Reagent A to form a compound of Formula (I):

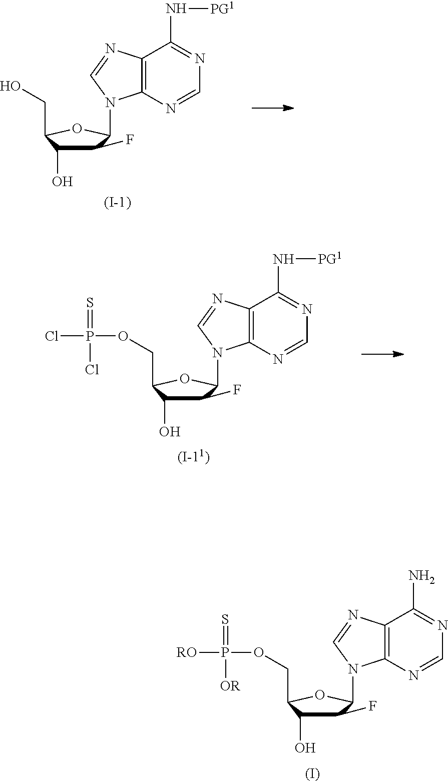

wherein
a) R is H;
b) PG¹ is selected from the group consisting of H, isobutyryl, pivaloyl, benzoyl, acetyl, octanoyl, 2-ethyl-hexanoyl;
c) the thiophosphorylating agent is selected from the group consisting of PSCl₂OK and PSCl₃;
d) the Catalyst A is selected from the group consisting of N-methyl morpholine, N-methyl imidazole, N-methyl-benzimidazole, quinine,

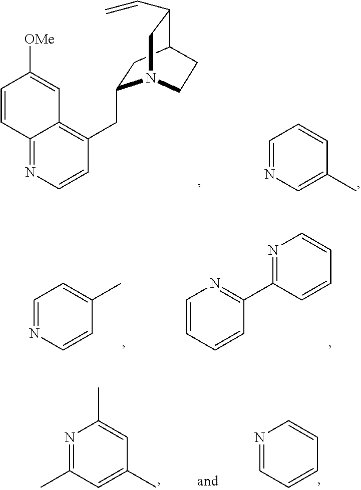

and mixtures thereof;
e) the at least one Base A is selected from the group consisting of 2,6-lutidine, pyridine, 4-picoline, pyridine, 2-picoline, quinoline, 2-F-pyridine, 2,4-lutidine, 2-methyl-pyridine, 2,4,6-trimethylpyridine, 2,3,5-trimethylpyridine, 3-methoxy-pyridine, 4- methyl-pyridine, quinuclidine, Hunig's base, triethylamine, 3-methyl-pyridine, and 2,6-di-tert-butyl-4-methyl pyridine, N-methyl morpholine, and mixtures thereof;
f) the at least one Solvent A is selected from the group consisting of THF, MeCN, acetone, DMPU, HFIP, TFE, glyme, DME, DMAc, propylene carbonate, tetraglyme, trimethyl phosphate, triethyl phosphate, 2-Me-THF, EtOAc, and MIBK, and mixtures thereof; and
g) the at least one Quenching Reagent A is selected from the group consisting of water, water in combination with pyridine, or water in combination with one or more additives.

In aspects of the fourth embodiment, the process further comprises isolating the compound of Formula (I) by crystallization from at least one Solvent B is selected from the group of water, methanol, ethanol, isopropanol and mixtures thereof. In instances of such aspects, the Solvent B is water. In specific instances of such aspects, the at least one Solvent B is provided in an amount in a range of from about 0.5 to about 20 volumes with respect to the amount of the compound of Formula (I-1), such as an amount in a range of from about 2 to about 15 volumes, or an amount of about 9 volumes.

In a first aspect of the fourth embodiment, Basel is thymine.

In a second aspect of the fourth embodiment, compound (I-3) is selected from the group consisting of 2′-deoxynucleosides. In instances of this aspect, the compound (I-3) is selected from the group consisting of thymidine, 2′-deoxyluridine, 2′-deoxycytidine, 2′-deoxyguanosine, and 2′-deoxyinosine. In particular instances of this aspect, the compound (I-3) is thymidine.

In a third aspect of the fourth embodiment, PG²-X is selected from the group consisting of acyl halides, alkyl halides, and silyl halides; and PG³-X is selected from the group consisting of acyl halides, alkyl halides, and silyl halides. In instances of this aspect, PG²-X is selected from the group consisting of isobutyryl chloride, pivaloyl chloride, trityl chloride, tert-butyldiphenylsilyl chloride, tert-butyldimethylsilyl chloride, triisopropylsilyl chloride, and trimethylsilyl chloride, and PG³-X is selected from the group consisting of trityl chloride, tert-butyldiphenylsilyl chloride, tert-butyldimethylsilyl chloride, triisopropylsilyl chloride, and trimethylsilyl chloride. In specific instances of this aspect, PG²-X is tert-butyldimethylsilyl chloride, and PG³-X is tert-butyldimethylsilyl chloride. In additional specific instances of this aspect, PG²-X is trimethylsilyl chloride, and PG³-X is trimethylsilyl chloride. In more specific instances of this aspect, PG²-X and PG³-X are provided in a combined amount in a range of from about 2.0 to about 3.0 equivalents with respect to the amount of the compound of Formula (I-3), or an amount of about 2.1 equivalents.

In aspects of the fourth embodiment, the preparation of the compound of Formula (I-2) from a compound of Formula (I-3) is accomplished by one step that also includes the protection of hydroxyl groups with PG² and PG^{3,} such as when PG² and PG³ are each trimethylsilyl, or may be accomplished in a series of steps.

In a fourth aspect of the fourth embodiment, the at least one Base D is selected from the group consisting of amines, and mixtures thereof. In instances of this aspect, the at least one Base D is selected from the group consisting of Hunig's Base, imidazole, pyridine, NMI, 2,6-lutidine, 2,4,6-collidine, DBU, DABCO, tetramethylguanidine, triethylamine, diisopropylethylamine, and mixtures thereof. In specific instances of this aspect, the at least one Base D is imidazole. In specific instances of this aspect, the at least one Base D is provided in an amount in a range of from about 1.0 to about 5.0 equivalents with respect to the amount of the compound of Formula (I-3), or an amount of about 3.0 equivalents.

In a fifth aspect of the fourth embodiment, the at least one Silylating Reagent B is selected from the group consisting of 1,3-bis(trimethylsilyl)urea, 3-(trimethylsilyl)-oxazolidin-2-one, hexamethyldisilazane, bistrimethylsilyl acetamide, bistrimethylsilyl trifluoroacetamide, and N-trimethylsilylimidazole, and mixtures thereof. In specific instances of this aspect, the at least one Silylating Reagent B is bistrimethylsilyl acetamide. In specific instances of this aspect, the at least one Silylating Reagent B is provided in an amount in a range of from about 2.0 to about 5.0 equivalents with respect to the amount of the compound of Formula (I-3), or an amount of about 4.5 equivalents.

In a sixth aspect of the fourth embodiment, the at least one Catalyst B is selected from the group consisting of Bronsted acid catalysts, and mixtures thereof. In instances of this aspect, the at least one Catalyst B is selected from the group consisting of mineral acids, sulfonic acids, sulfonimides, N-acylsulfonamides, electron poor sulfonamides, dialkyl-phosphine sulfides, dialkyl- phosphine selenides, diaryl-phosphine sulfides, diaryl-phosphine selenides, thio- and seleno-phosphinic and thio- and seleno-phosphoric acids, bis(thiophosphoryl)amides and bis(selenophosphoryl)amides, and bis(thiophosphoryl)amides and bis(selenophosphoryl)amides. In specific instances of this aspect, the at least one Catalyst B is selected from the group consisting of sulfuric acid, methanesulfonic acid, N,N-bistriflimide, 1,2-phenyldisulfonimide, N,N-dibenzenesulfonimide, N,N-bis(4-methoxybenzenesulfonyl)amide, N-(4-chlorobenzenesulfonyl)-N-methanesulfonylamide, N-benzenesulfonyl-benzamide, N,N-bis(methanesulfonyl)amide, saccharin, thiosaccharin, 6-nitrosaccharin, 6-chlorosaccharin, 5-fluorosaccharin, perfluorobenzenesulfonamide, diphenyldithiophosphinic acid, diethyldithiophosphoric acid, N,N-bis(diphenylthiophosphoryl)amide, and N,N-bis(diphenylselenophosphoryl)amide, and mixtures thereof. In more specific instances of this aspect, the at least one Catalyst B is selected from the group consisting of DBSI, PTPI, and PSePI, and mixtures thereof. In other specific instances of this aspect, the at least one Catalyst B is provided in an amount in a range of from about 0.001 to about 0.1 equivalents with respect to the amount of the compound of Formula (I-3), or an amount of about 0.01 equivalents.

In a seventh aspect of the fourth embodiment, said reacting is performed in the presence of at least one Solvent F, which is selected from the group consisting of hydrocarbons, halocarbons, ethers, and silanes, and mixtures thereof. In instances of this aspect, the at least one Solvent F is selected from the group consisting of dichloromethane, dichloroethane, hexane, heptane, cyclohexane, CPME, toluene, trifluorotoluene, hexamethyldisiloxane, and hexamethyldisilazne, and mixtures thereof. In specific instances of this aspect, the at least one Solvent F is selected from the group consisting of heptane, toluene, and mixtures thereof. In specific instances of this aspect, the at least one Solvent F is provided in an amount in a range of from about 0 to about 20 volumes with respect to the amount of the compound of Formula (I-3), or an amount of about 10 volumes.

In an eighth aspect of the fourth embodiment, the compound of Formula (I-2) is purified by adding the reaction mixture to an alcohol, such as methanol, ethanol, 2-propanol, or a mixture of alcohols, to induce selective alcoholysis and precipitation of by-products. In instances of this aspect, the at least one alcohol is provided in an amount in a range of from about 4 to about 20 molar equivalent with respect to the amount of the compound of Formula (I-3). In a more specific instance of this aspect, the at least one alcohol is 2-propanol.

In a ninth aspect of the fourth embodiment, the at least one Fluorinating Agent A is selected from the group consisting of N-fluorobenzenesulfonimide, 1-chloromethyl-4-fluoro-1,4-diazoniabicyclo[2.2.2]octane bis(tetrafluoroborate), 1-fluoro-4-methyl-1,4-diazoniabicyclo [2.2.2] octanebis(tetrafluoroborate), N-fluoropyridinium triflate, and N-fluoropyridinium tetrafluoroborate, and mixtures thereof. In specific occurrences of this instance, the at least one

Fluorinating Agent A is N-fluorobenzenesulfonimide. In additional occurrences of this instance, the Fluorinating Agent A is provided in an amount in a range of from about 1.10 to about 2.0 equivalents with respect to the amount of the compound of Formula (I-2), or an amount of about 1.10 equivalents.

In a tenth aspect of the fourth embodiment, the at least one Base B is selected from the group consisting of pyridine, 2,6-lutidine, triethylamine, N-methylmorpholine, 2,4,6-collidine, potassium carbonate, dibasic potassium phosphate, tribasic potassium phosphate, and sodium bicarbonate, and mixtures thereof. In occurrences of this instance, the at least one Base B is 2,6-lutidine. In additional occurrences of this instance, the at least one Base B is provided in an amount in a range of from about 0.1 to about 0.9 equivalents with respect to the amount of the compound of Formula (I-2), or an amount of about 0.5 equivalents.

In an eleventh aspect of the fourth embodiment, the at least one Silylating Reagent A is selected from the group consisting of 1,3-bis(trimethylsilyl)urea, 3-(trimethylsilyl)-oxazolidin-2-one, hexamethyldisilazane (HDMS), bistrimethylsilyl acetamide (BSA), bistrimethylsilyl trifluoroacetamide (BSTFA, also referred to as trimethylsilyl 2,2,2-trifluoro-N-(trimethylsilyl)acetimidate), and N-trimethylsilylimidazole, and mixtures thereof. In occurrences of this instance, the at least one Silylating Reagent A is bistrimethylsilyl trifluoroacetamide. In additional occurrences of this instance, the Silylating Reagent A is provided in an amount in a range of from about 0.05 to about 2.0 equivalents with respect to the amount of the compound of Formula (I-1), or an amount of about 0.05 equivalents.

In a twelfth aspect of the fourth embodiment, the at least one Solvent C is selected from the group consisting of ethyl acetate, dioxane, dimethoxyethane, tetrahydrofuran, 2-methyltetrahydrofuran, cyclopentyl methylether, methyl tert-butyl ether, 1,2-dichloroethane, acetonitrile, isopropyl acetate, diethylcarbonate, and toluene, and mixtures thereof. In occurrences of this instance, the at least one Solvent C is toluene. In additional occurrences of this instance, the Solvent C is provided in an amount in a range of from about 5 to about 20 volumes with respect to the amount of the compound of Formula (I-2), or an amount of about 5 volumes.

In a thirteenth aspect of the fourth embodiment, the at least one Base C is selected from the group consisting of N,N-diisopropylethylamine, 2,6-lutidine, 2,2,6,6-tetramethylpiperidine, potassium acetate, potassium tert-butoxide, potassium carbonate, tribasic potassium phosphate, N-methylmorpholine, potassium bicarbonate, sodium bicarbonate, sodium carbonate, cesium carbonate, DBU, 2,4,6-collidine, DABCO, N-methylimidazole, 2,6-ditert-butyl-4-methyl pyridine, potassium tert-butoxide, and potassium hexamethyldisilazide, and mixtures thereof. In occurrences of this instance, the at least one Base C is 2,6-lutidine. In additional occurrences of this instance, the at least one Base C is provided in an amount in a range of from about 0.75 to about 3.0 equivalents with respect to the amount of the compound of Formula (I-2¹), or an amount of about 1.2 equivalents.

In a fourteenth aspect of the fourth embodiment, the at least one Solvent D is selected from the group consisting of toluene, tetrahydrofuran, acetonitrile, dimethylformamide, acetone, dimethylacetamide, cyclopentyl methyl ether, dimethoxyethane, diethylcarbonate, 2-methyl tetrahydrofuran, isopropyl acetate, and ethyl acetate, and mixtures thereof. In particular occurrences of this instance, the at least one Solvent D is ethyl acetate. In additional occurrences of this instance, the at least one Solvent D is provided in an amount in a range of from about 5 to about 30 volumes with respect to the amount of the compound of Formula (I-2¹), or an amount of about 20 volumes.

In a fifteenth aspect of the fourth embodiment, the at least one Acid A is selected from the group consisting of TFA, HCl, H₂SO₄, MsOH, TsOH, and mixtures thereof. In occurrences of this instance, the at least one Acid A is TFA. In specific instances of this aspect, the at least one Acid A is provided in an amount in a range of from about 0.1 to about 8.0 equivalents with respect to the amount of the compound of Formula (I-2²), or an amount of about 0.1 equivalents.

In a sixteenth aspect of the fourth embodiment, the at least one Solvent E is selected from the group consisting of isopropanol, methanol, water, ethyl acetate, and ethanol, and mixtures thereof. In particular occurrences of this instance, the at least one Solvent E is ethanol. In additional instances of this aspect, the at least one Solvent E is provided in an amount in a range of from about 0.5 to about 5.0 volumes with respect to the amount of the compound of Formula (I-2²), or an amount of about 0.5 volumes.

In a seventeenth aspect of the fourth embodiment, the thiophosphorylating agent is selected from the group consisting of PSCl₂OK and PSCl₃. In instances of this aspect of this first aspect of the first embodiment, the thiophosphorylating agent is PSCl₃. In specific instances of this aspect, the thiophosphorylating agent is provided in an amount in a range of from about 0.5 to about 5.0 equivalents with respect to the amount of the compound of Formula (I-1), such as an amount in a range of from about 0.75 to about 3.5 equivalents, an amount in a range of from about 1.0 to about 2.5 equivalents, or an amount in a range of from about 1.5 to about 2.0 equivalents.

In an eighteenth aspect of the fourth embodiment, the at least one Catalyst A is selected from the group consisting of N-methyl morpholine, N-methyl imidazole, N-methyl-benzimidazole, quinine,

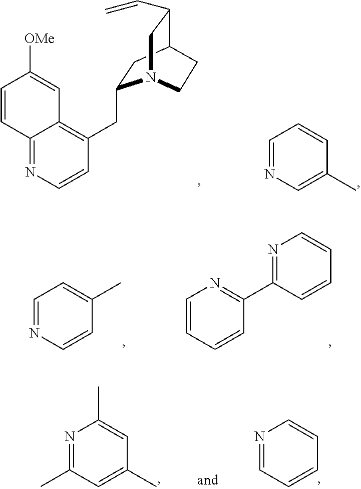

and mixtures thereof. In instances of this aspect, the at least one Catalyst A is selected from

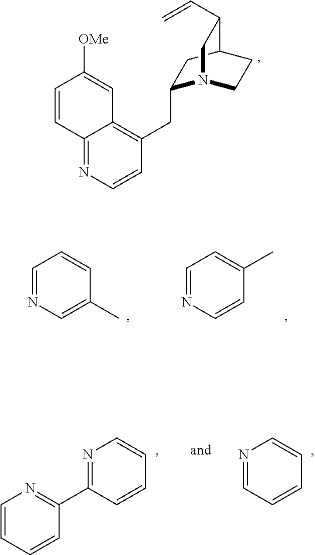

and mixtures thereof. In specific instances of this aspect, the at least one Catalyst A is

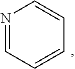

In specific instances of this aspect, the at least one Catalyst A is provided in an amount in a range of from about 0.01 to about 5.0 equivalents with respect to the amount of the compound of Formula (I-1), such as an amount in a range of from about 0.1 to about 1.0 equivalents, an amount in a range of from about 0.15 to about 0.4 equivalents, or an amount of about 0.25 equivalents.

In a nineteenth aspect of the fourth embodiment, the at least one Base A is selected from the group consisting of 2,6-lutidine, pyridine, 4-picoline, pyridine, 2-picoline, quinoline, 2-F-pyridine, 2,4-lutidine, 2-methyl-pyridine, 2,4,6-trimethylpyridine, 2,3,5-trimethylpyridine, 3-methoxy-pyridine, 4-methyl-pyridine, quinuclidine, Hunig's base, triethylamine, 3-methyl-pyridine, and 2,6-di-tert-butyl-4-methyl pyridine, N-methyl morpholine and mixtures thereof. In specific instances of this aspect, the at least one Base A is selected from the group consisting of 2,6-lutidine, 2,4,6-trimethylpyridine, 2,3,5-trimethylpyridine, 2,4-dimethylpyridine, and pyridine. In still more specific instances of this aspect, the at least one Base A is 2,6-lutidine. In specific instances of this aspect, the at least one Base A is provided in an amount in a range of from about 0.5 to about 5.0 equivalents with respect to the amount of the compound of Formula (I-1), such as an amount in a range of from about 1.0 to about 3 equivalents, or an amount of about 1.5 equivalents.

In a twentieth aspect of the fourth embodiment, the at least one Solvent A is selected from the group consisting of THF, MeCN, acetone, DMPU, HFIP, TFE, glyme, DME, DMAc, propylene carbonate, tetraglyme, trimethyl phosphate, triethyl phosphate, 2-Me-THF,

EtOAc, and MIBK, and mixtures thereof. In instances of this aspect, the at least one Solvent A is selected from the group consisting of tetraglyme, MeCN, trimethyl phosphate, and triethyl phosphate, and mixtures thereof. In specific instances of this aspect, the at least one Solvent A is selected from triethyl phosphate and tetraglyme, and mixtures thereof. In particular specific instances of this aspect, the at least one Solvent A is triethyl phosphate. In specific instances of this aspect, the at least one Solvent A is provided in an amount in a range of from about 3 to about 50 volumes with respect to the amount of the compound of Formula (I-1), such as an amount in a range of from about 3 to about 20 volumes, or an amount of about 5 volumes.

In a twenty-first aspect of the fourth embodiment, the reacting to form the compound of Formula (I-1¹) is conducted at a temperature in a range of from about −20° C. to about 30° C., such as in a range of from about −10° C. to about 10° C., or about −5° C.

In a twenty-second aspect of the fourth embodiment, the reaction forming the compound of Formula (I-1¹) is quenched from at least one Quenching Reagent A is selected from the group consisting of water, water in combination with pyridine, or water in combination with one or more additives, where said additives are independently is selected from guanidine-HCl, phenol, sodium dodecyl sulfate, thiourea, lithium acetate, magnesium chloride, and urea, and mixtures thereof. In specific instances of this aspect, the at least one Quenching Reagent A is water. In specific instances of this aspect, the at least one Quenching Reagent A is provided in an amount in a range of from about 0.5 to about 20 volumes with respect to the amount of the compound of Formula (I-1), such as an amount in a range of from about 0.5 to about 5 volumes, or an amount of about 2 volumes.

In a twenty-third aspect of the fourth embodiment, the reaction forming the compound of Formula (I) is conducted by heating the reaction at a temperature in a range of from about 20° C. to about 100° C., such as at a temperature in a range of from about 30° C. to about 60° C., or about 50° C. In specific instances of this aspect, the reaction is aged for a duration in a range of from about 30 mins to 20 h, such as a duration in a range of from about 1 h to 5 h, or a duration of about 3 h.

In a twenty-third aspect of the fourth embodiment, the process further comprises isolating the compound of Formula (I) by crystallization from at least one Solvent B is selected from the group consisting of water, methanol, ethanol, isopropanol, and mixtures thereof. In more specific instances of this aspect, the Solvent B is water. In specific instances of this aspect, the at least one Solvent B is provided in an amount in a range of from about 0.5 to about 20 volumes with respect to the amount of the compound of Formula (I-1), such as an amount in a range of from about 2 to about 15 volumes, or an amount of about 9 volumes.

In aspects of the fourth embodiment, the process further comprises forming a sodium or potassium salt of the compound of Formula (I).

In a fifth embodiment, the disclosure provides a process for the preparation of compounds of Formula (Ia), or a pharmaceutically acceptable salt, hydrate, or solvate thereof:

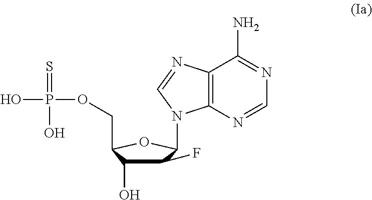

comprising
i) reacting thymidine with trimethylsilyl chloride in the presence of imidazole, bistrimethylsilyl acetamide, at least one Catalyst B is selected from the group consisting of N,N-dibenzenesulfonimide, N,N-bis(diphenylthiophosphoryl)amide, and N,N-bis(diphenylselenophosphoryl) amide, and mixtures thereof, and at least one Solvent F, is selected from the group consisting of dichloromethane, dichloroethane, hexane, heptane, cyclohexane, CPME, toluene, trifluorotoluene, hexamethyldisiloxane, and hexamethyldisilazne, and mixtures thereof:

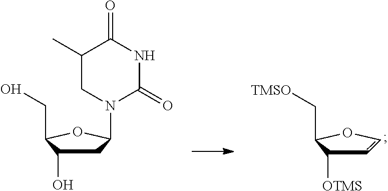

ii) reacting the product of step i) successively
a) with N-fluorobenzenesulfonimide in the presence of 2,6-lutidine, bistrimethylsilyl trifluoroacetamide, and toluene,
b) with pivaloyl-protected adenine in the presence of 2,6-lutidine and ethyl acetate,
c) with TFA in the presence of ethanol:

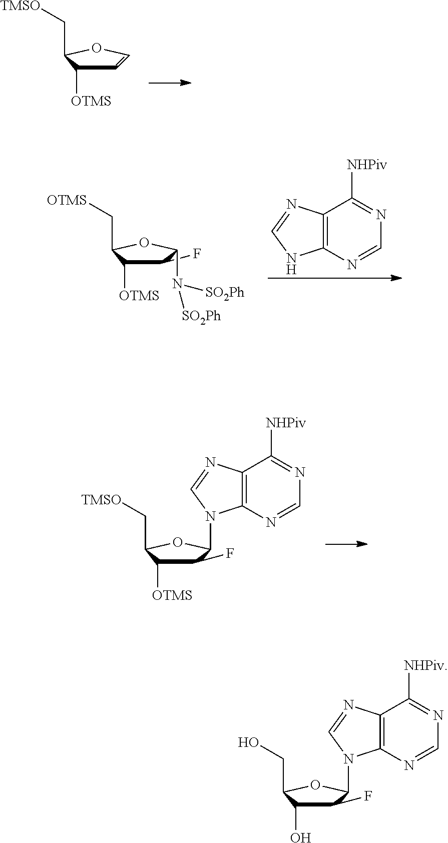

In aspects of the fifth embodiment, the process further comprises forming a sodium or potassium salt of the compound of Formula (Ia).

The fourth through fifth embodiments are understood to include and incorporate the first through third embodiments in all their aspects.

In a sixth embodiment, the disclosure provides compounds is selected from the group consisting of:

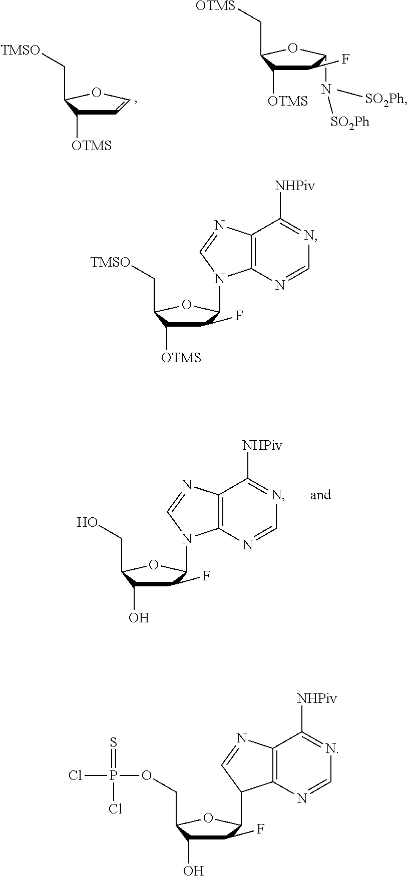

Methods for preparing the compound of Formula (I) as well as synthetic intermediates useful for its preparation according to the invention are exemplified below. Starting materials are made according to procedures known in the art or as illustrated herein. The following examples are provided so that the invention may be more fully understood. These examples are illustrative only and should not be construed as limiting the invention in any way.

Nuclear magnetic resonance (NMR) spectra were recorded for ¹H NMR at 500 MHz or 400 MHz. Chemical shifts were reported in ppm relative to the residual deuterated solvent for ¹H. Splitting patterns for ¹H NMR signals are designated as: (singlet), d (doublet), t (triplet), q (quartet), quint (quintuplet), broad singlet (br s) or m (multiplet).

### EXAMPLES

### Synthesis of Trimethyl(((2R,3S)-3-((trimethylsilyl)oxy)-2,3-dihydrofuran-2-yl)methoxy) silane from Thymidine

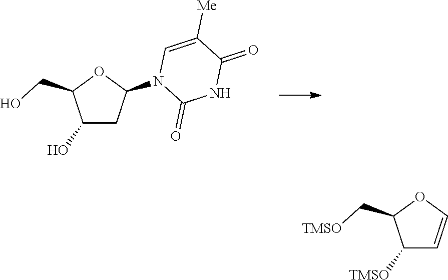

A 100 L vessel was charged with toluene (14.5 L), thymidine (4825 g, 20 mol), 2,6-lutidine (1081 g, 0.400 mol), PTPI (90 g, 0.200 mol) and heptane (33.8 L). The mixture was heated to 90° C. To this, BSA (17.4 kg, 85.6 mol) was added over 30 min. The mixture was heated to 100° C. and stirred at 100-107° C. for 3 h. After cooling to room temperature, the reaction mixture was transferred to another 100 L reactor containing i-PrOH (12.3 L, 161 mol) slowly (204 ml/min). Toluene (1 L) was used to rinse and transfer any remaining material in the first reactor. The resulting slurry was stirred at 35° C. for 2 h, then cooled to 10° C. and aged at that temperature overnight. The resulting slurry was filtered, and the filter cake was washed with heptane (20.0 L). The combined filtrates were passed through a plug of basic alumina and transferred to a 100 L vessel. The resulting solution was concentrated under vacuum to the total volume of 24 L, which was used in the subsequent reaction without further purification.

¹H NMR (400 MHz, CD₂Cl₂); δ 6.50 (dd, J=2.7, 1.1 Hz, 1H), 5.06 (t, J=2.7 Hz, 1H), 4.84 (td, J=2.7, 1.0 Hz, 1H), 4.28 (ddd, J=6.7, 6.1, 2.7 Hz, 1H), 3.67 (dd, J=10.6, 6.1 Hz, 1H), 3.47 (dd, J=10.6, 6.7 Hz, 1H), 0.17 (s, 9H), 0.16 (s, 9H).

### Synthesis of Trimethyl(((2R,3S)-3-((trimethylsilyl)oxy)-2,3-dihydrofuran-2-yl)methoxy) silane from 2′-Deoxyuridine

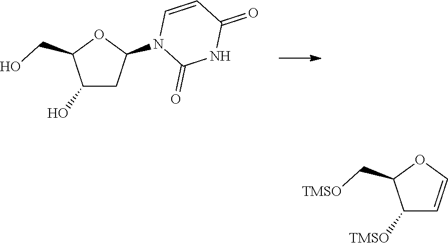

In an 8 mL vial, dry 2′-deoxyuridine (1 mmol), PTPI (0.01 eq, 5 mg), 2,6-lutidine (0.5 eq, 58 μL), 1 mL heptane, 1 mL toluene, and 3.5 eq. of BSA was added under nitrogen atmosphere. The reaction was stirred at 100° C. for 3 h. Reaction progress was monitored via HPLC by the presence of starting material.

### Synthesis of 2-tert-butyl(((2R,3S)-3-((tert-butyldimethylsilyl)oxy)-2,3-dihydrofuran-2-yl)methoxy)dimethylsilane (2-TBS)

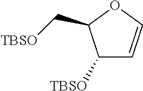 [SMILES: CC(C)(C)[Si](C)(C)OC[C@H]1OC=C[C@@H]1O[Si](C)(C)C(C)(C)C]

In a 2 L flask was charged ammonium sulfate (5.38 g, 40.7mmol), bis(tert-butyldimethylsilyl)thymidine (100 g, 203 mmol), and 2,6-di-tert-butyl-4-methylphenol (0.045 g, 0.203 mmol). HMDS (141 mL, 671 mmol) and heptane (1000 mL) were subsequently added, and the reaction mixture was heated to reflux (140° C. external bath) under nitrogen atmosphere. After 34 h, the reaction mixture was cooled to ambient temperature. 2,4,6-trimethylpyridine (13.55 mL, 102 mmol) was added followed by ethanol (35.6 mL, 610 mmol) via syringe pump over 2 h. The resulting slurry was then filtered, and the cake was washed with CPME (4×150 mL). The filtrate was concentrated to provide 2-TBS (57.14 g, 166 mmol,) by quantitative NMR analysis.

¹H NMR (500 MHz, CDCl₃) δ 6.47 (dd, J=2.6, 0.8 Hz, 1H), 5.01 (t, J=2.6 Hz, 1H), 4.87 (td, J=2.6, 0.8 Hz, 1H), 4.29 (td, J=6.0, 2.8 Hz, 1H), 3.69 (dd, J=10.7, 5.7 Hz, 1H), 3.51 (dd, J=10.7, 6.3 Hz, 1H), 0.90 (s, 9H), 0.89 (s, 9H), 0.09 (s, 3H), 0.09 (s, 3H), 0.07 (s, 3H), 0.06 (s, 3H); ¹³C NMR (126 MHz, CDCl₃) δ 6 149.1, 103.6, 89.1, 76.1, 63.0, 26.1, 26.0, 18.5, 18.2, −4.1, −4.3, −5.2, −5.2.

### Synthesis of N-(2S,3S,4R,5R)-4-((tert-butyldimethylsilyl)oxy)-5-(((tert-butyldimethylsilyl) oxy)methyl)-3-fluorotetrahydrofuran-2-yl)-N-(phenylsulfonyl)benzenesulfonamide (3- TBS)

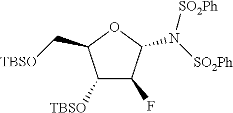

In a 1 L flask was charged the crude 2-TBS (1.0 equiv, 82.95 g, 166 mmol) and CPME (263 mL). Additionally, 2,4,6-trimethylpyridine (4.53 mL, 34.2 mmol), BSTFA (22.02 mL, 83 mmol), and NFSI (68.0 g, 216 mmol) were added to the reaction mixture. The resulting mixture was warmed to 65° C. and stirred for 20 h. After cooling to ambient temperature, heptane (286 mL) was added, and the reaction mixture was stirred for 1.75 h at ambient temperature. The resulting slurry was filtered, and the cake was washed with CPME:heptane (1:1, 286 mL). The filtrate was subsequently concentrated under vacuum. Heptane (286 mL) was added to the concentrated crude material, and the mixture was heated to 70° C. The mixture was filtered while hot into a 1 L flask, and the filtrate was crystallized while being slowly cooled to ambient temperature. The resulting slurry was further cooled to −30 to −35° C. and filtered. After drying under vacuum, the desired DBSI adduct 3-TBS (94.63 g, 138 mmol) was collected.

¹H NMR (500 MHz, CDCl₃) δ 8.04 (dd, J=8.5, 1.1 Hz, 4H), 7.64 (t, J=7.5 Hz, 2H), 7.54 (t, J=7.9 Hz, 4H), 6.00 (dd, J=16.5, 5.9 Hz, 1H), 5.67 (ddd, J=57.2, 6.3, 6.3 Hz, 1H), 4.48 (ddd, J=21.3, 8.9, 6.7 Hz, 1H), 4.39-4.24 (m, 1H), 3.78 (ddd, J=12.0, 1.8, 1.8 Hz, 1H), 3.65 (dd, J=12.0, 3.1 Hz, 1H), 0.92 (s, 9H), 0.88 (s, 9H), 0.10 (s, 3H), 0.08 (s, 3H), 0.05 (s, 3H), 0.05 (s, 3H); ¹³C NMR (126 MHz, CDCl₃) 6 140.6, 134.0, 129.1, 128.4, 99.7 (d, J=188.1 Hz), 92.2 (d, J=36.8 Hz), 83.4 (d, J=9.9 Hz), 73.0 (d, J=20.9 Hz), 61.3, 26.0, 25.7, 18.5, 18.0, −4.5, −5.0, −5.1, −5.3; ¹⁹F NMR (500 MHz, CDCl₃) δ −195.0.

### Synthesis of 1-((2R,4S,5R)-4-((tert-butyldimethylsilyl)oxy)-5-(((tert-butyldimethylsilyl)oxy) methyl)tetrahydrofuran-2-yl)-5-methyloyrimidine-2,4(1H,3H)-dione

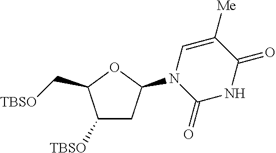 [SMILES: Cc1cn([C@H]2C[C@H](O[Si](C)(C)C(C)(C)C)[C@@H](CO[Si](C)(C)C(C)(C)C)O2)c(=O)[nH]c1=O]

While under a nitrogen atmosphere, thymidine (12.1 g, 50 mmol), imidazole (2.5 equiv, 8.5 g, 125 mmol), tert-butyldimethylsilyl chloride (2.2 equiv, 16.6 g, 110 mmol), DMF (20 mL), and DMAP (0.01 equiv, 0.061 g, 0.5 mmol) were added to a 200 mL round-bottom flask, and the resulting mixture was stirred for 1 h at ambient temperature. The reaction was determined to be complete by HPLC. Subsequent addition of 100 mL water was followed by stirring at ambient temperature for 1 h. Filtration of the slurry was performed, and the cake was washed with 200 mL water. The cake was dissolved in 100 mL MTBE, and the solution was washed with 100 mL water and dried over magnesium sulfate. The filtered MTBE solution was evaporated to approximately 30 mL, diluted with 30 mL hexanes and 80 mL heptane and evaporated to approximately 100 mL. The residue was cooled to 0° C. over 2 h, and crystallization was observed to occur. The slurry was filtered and washed with 30 mL 9:1 hexanes:MTBE and subsequently with 50 mL hexanes. The solid was dried under a nitrogen stream to provide 1-((2R,4S,5R)-4-((tert-butyldimethylsilyl) oxy)-5-(((tert-butyldimethylsilyl)oxy)methyl) tetrahydrofuran-2-yl)-5-methylpyrimidine-2,4(1H,3H)-dione (21 g, 44.6 mmol).

¹H NMR (500 MHz, CDCl₃) 6 8.35 (s, 1H), 7.47 (d, J=1.2 Hz, 1H), 6.33 (dd, J=7.9, 5.8 Hz, 1H), 4.40 (ddd, J=5.6, 2.5, 2.5 Hz, 1H), 3.93 (ddd, J=2.5, 2.5, 2.5 Hz, 1H), 3.87 (dd, J=11.4, 2.6 Hz, 1H), 3.76 (dd, J=11.4, 2.4 Hz, 1H), 2.25 (ddd, J=13.1, 5.8, 2.6 Hz, 1H), 2.00 (ddd, J=13.3, 7.9, 6.1 Hz, 1H), 1.92 (d, J=1.1 Hz, 3H), 0.93 (s, 9H), 0.89 (s, 9H), 0.11 (s, 3H), 0.11 (s, 3H), 0.08 (s, 3H), 0.07 (s, 3H); ¹³C NMR (126 MHz, CDCl₃) δ 164.3, 150.7, 135.8, 111.2, 88.2, 85.2, 72.6, 63.4, 41.8, 26.3, 26.1, 18.8, 18.4, 12.9, −4.3, −4.5, −5.0, −5.1.

### Synthesis of (O—{[(2R,3R,4S,5R)-5-(6-amino-9H-purin-9-yl)-4-fluoro-3-hydroxyoxolan-2-yl]methyl}O,O-dihydrogen phosphorothioate

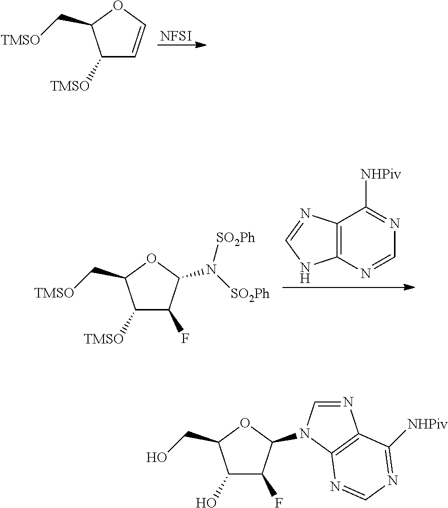

To a 100 L reactor was charged the crude toluene stream for trimethyl(((2R,3S)-3-((trimethylsilyl)oxy)-2,3-dihydrofuran-2-yl)methoxy)silane (10.2 kg, 13.4 mol), which contained 0.36 equivalents lutidine and 2 vol toluene. To this was added toluene (8.75 L), 2,6-lutidine (0.563 L, 4.84 mol), and BSTFA (0.178 L, 0.672 mol), and the resulting mixture was warmed to 65° C. N-fluorobenzenesulfonimide (NFSI) (4.66 kg, 14.77 mol) was added portionwise, then toluene (1.75 L) was used to rinse the sides of the reactor. The reaction mixture was stirred at 65° C. until trimethyl(((2R,3S)-3-((trimethylsilyl)oxy)-2,3-dihydrofuran-2-yl)methoxy)silane was consumed judged by NMR analysis, after which 2,6-lutidine (0.782 L, 6.72 mol), ethyl acetate (50.75 L) and N-(9H-purin-6-yl)pivalamide (2.88 kg, 12.76 mol) were added. An additional 1.75 L of ethyl acetate was used to rinse the sides of the reactor. The resulting mixture was warmed to 75° C. and stirred for overnight. The crude reaction mixture was then concentrated under vacuum to a total volume of 35 L. The resulting slurry was filtered, and the filter cake was washed with ethyl acetate (17.5 L, 5 vol). The filtrate was transferred to a 50 L reactor while continuously evaporating under vacuum to a total volume of 17.5 L. To this, ethanol (5.25 L), 2,6-lutidine (0.313 L, 2.69 mol), and TFA (103 ml, 1.34 mol) were added to start desilylation. Vacuum distillation while feeding 21 L of 3.8:1 v/v EtOAc:EtOH was conducted to aid the desilylation. When the desilylation was achieved with >90% conversion 17.5 L EtOAc was fed into the reactor while distilling away excess EtOH. An additional continuous vacuum distillation was performed with 3.5 L toluene feed while the mixture was concentrated to the total volume of 17.5 L. After the distillation was completed, the reaction mixture was stirred at room temperature overnight to crystallize. The product was collected by filtration rinsing with 21 L of 2:10:88 v/v/v EtOH:tol:EtOAc. Total mass was 3.16 kg.

¹H NMR (500 MHz, DMSO-d6) δ 10.19 (s, 1H), 8.72 (s, 1H), 8.56 (d, J=1.9 Hz, 1H), 7.30-7.10 (toluene, m, 5H), 6.55 (dd, J=13.4, 4.7 Hz, 1H), 5.99 (bs, 1H), 5.29 (ddd, J=52.6, 4.3, 4.3 Hz, 1H), 4.49 (ddd, J=19.1, 4.6, 4.6, 1H), 3.89 (ddd, J=4.8, 4.8, 4.8 Hz, 1H), 3.72 (dd, J=12.1, 3.1 Hz, 1H), 3.66 (dd, J=12.0, 5.1 Hz, 1H), 2.30 (toluene, s, 3H), 1.28 (s, 9H); ¹³C NMR (124 MHz, DMSO-d6) δ 176.3, 151.8, 151.7, 150.4, 142.8 (d, J=3.8 Hz), 137.4 (toluene), 128.9 (toluene), 128.2 (toluene), 125.2 (toluene), 125.1, 95.4 (d, J=192.4 Hz), 83.5 (d, J=5.7 Hz), 81.5 (d, J=17.0 Hz), 72.5 (d, J=23.0 Hz), 60.3, 26.9, 21.0 (toluene); ¹⁹F NMR (470 MHz, DMSO-d6) 6 197.9.

### Synthesis of (O—{[(2R,3R,4S,5R)-5-(6-amino-9H-purin-9-yl)-4-fluoro-3-hydroxyoxolan-2-yl]methyl}O,O-dihydrogen phosphorothioate from (2R,3R,4R,5R)-5-(6-amino-9H-purin-9-yl)-4-fluoro-2-(hydroxymethyl)tetrahydrofuran-3-ol

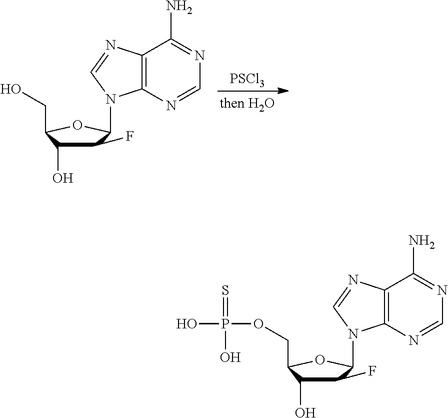

(2R,3R,4R,5R)-5-(6-amino-9H-purin-9-yl)-4-fluoro-2-(hydroxymethyl)tetrahydrofuran-3-ol (50 g, 186 mmol) was azeotroped with 3×100 mL of dry pyridine and was dissolved in 500 mL (10 vol) of dry pyridine (KF=128 ppm). The pyridine solution was cooled to 0° C. for 1 h. Thiophosphoryl chloride (1.04 eq) was added dropwise at 0° C. over 10 min. The reaction was stirred at 0° C. for 80 min, with constant monitoring by UPLC. The reaction was filtered to remove the excess starting material. Water (10 eq) was then added to the filtrate at 0° C. and was slowly warmed to room temperature. The reaction was allowed to stir for an additional 30 min at room temperature. The volatiles were removed in vacuo, and the product was dissolved in 500 mL of water. The solution pH was 4. The solution was filtered, and the filtrate was stirred while 12M HCl was added until the pH of the solution was 0 (about 35 mL). The resulting slurry was allowed to stir at room temperature overnight (˜16 h). Then the slurry was allowed to settle for 1 h. The slurry was then filtered, and the filter cake was washed with 200 mL of water. The washed cake was allowed to dry over a stream of nitrogen overnight (29.9 g).

¹H NMR (500 MHz, DMSO-d₆) δ=8.26 (d, J=1.9 Hz, 1H), 8.21 (s, 1H), 7.55 (br s, 2H), 6.46 (dd, J=15.0, 4.5 Hz, 1H), 5.24 (dt, J=52.4, 4.1 Hz, 1H), 4.51 (dt, J=18.1, 4.2 Hz, 1H), 4.22-4.04 (m, 3H); ¹³C NMR (125 MHz, DMSO-d6): δ=155.9, 152.6, 149.5, 140.3 (d, J=4.1 Hz), 118.7, 95.4 (d, J=191.9 Hz), 82.1 (d, J=16.7 Hz), 81.8 (dd, J=9.3, 5.3 Hz), 73.5 (d, J=23.7 Hz), 65.50 (dd, J=4.6, 1.8 Hz); ¹⁹F NMR (470 MHz, DMSO-d6): δ=−197.80 (ddd, J=52.7, 16.6, 16.6 Hz, 1F); ³¹P NMR (202 MHz, DMSO-d6): δ=59.49 (dd, J=7.4, 7.4 Hz, 1P).

### Synthesis of (O—{[(2R,3R,4S,5R)-5-(6-amino-9H-purin-9-yl)-4-fluoro-3-hydroxyoxolan-2-yl]methyl}O,O-dihydrogen phosphorothioate from Piv-protected (2R,3R,4R,5R)-5-(6-amino-9H-purin-9-yl)-4-fluoro-2-(hydroxymethyl)tetrahydrofuran-3-ol

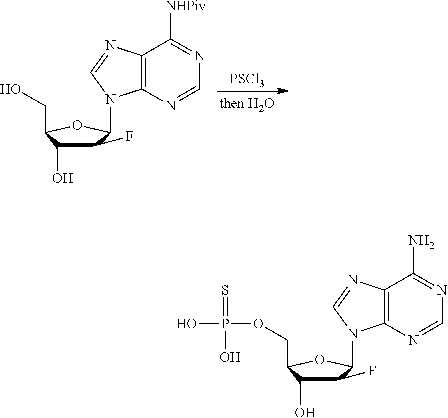

In a 50 L reactor was charged triethylphosphate (4vol, 8.58 L), Piv-protected (2R,3R,4R,5R)-5-(6-amino-9H-purin-9-yl)-4-fluoro-2-(hydroxymethyl)tetrahydrofuran-3-ol (2.5 kg, 85.75 wt %) followed by the remaining triethylphosphate (1 vol, 2.14 L) washing the sides of the vessel. To this, 2,6-lutidine (3 eq, 1.97 kg) and pyridine (0.3 eq, 144 g) were charged, and the resulting mixture was cooled to −20° C. Then thiophosphoryl chloride (1.835 kg, 1.75 eq.) was added slowly over 1 h. The reaction mixture was aged at −20° C. for overnight, after which additional thiophosphoryl chloride (32 mL, 0.05 eq.) was added. Water (8 eq, 0.87 L) was added dropwise over 1 h to quench the reaction. Additional water (32 eq, 3.5 L) was added dropwise over 1 h, then the resulting mixture was warmed to 50° C. and aged at that temperature for 3 h. After pivaloyl group was removed judged by HPLC analysis, the mixture was cooled to 30° C., and added water (9 vol, 19.3 L) to crystallize the product while cooling to 0° C. slowly. The product was collected by filtration rinsing with water (12.5 L) then dried under vacuum with nitrogen sweep. The resulting product (1.971 kg, 88.65 wt %) was then collected and stored under ambient temperature.

¹H NMR (500 MHz, DMSO-d₆) δ=8.26 (d, J=1.9 Hz, 1H), 8.21 (s, 1H), 7.55 (br s, 2H), 6.46 (dd, J=15.0, 4.5 Hz, 1H), 5.24 (dt, J=52.4, 4.1 Hz, 1H), 4.51 (dt, J=18.1, 4.2 Hz, 1H), 4.22-4.04 (m, 3H); ¹³C NMR (125 MHz, DMSO-d6): δ=155.9, 152.6, 149.5, 140.3 (d, J=4.1 Hz), 118.7, 95.4 (d, J=191.9 Hz), 82.1 (d, J=16.7 Hz), 81.8 (dd, J=9.3, 5.3 Hz), 73.5 (d, J=23.7 Hz), 65.50 (dd, J=4.6, 1.8 Hz); ¹⁹F NMR (470 MHz, DMSO-d6): δ=−197.80 (ddd, J=52.7, 16.6, 16.6 Hz, 1F); ³¹P NMR (202 MHz, DMSO-d6): δ=59.49 (dd, J=7.4, 7.4 Hz, 1P).

It will be appreciated that various of the above-discussed and other features and functions, or alternatives thereof, may be desirably combined into many other different systems or applications. It will also be appreciated that various presently unforeseen or unanticipated alternatives, modifications, variations or improvements therein may be subsequently made by those skilled in the art, and these are also intended to be encompassed by the following claims.

1. A process for preparing a compound of Formula (I): 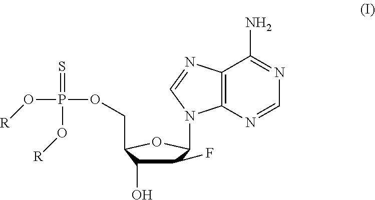
wherein each R is independently selected from the group consisting of H, Na, and K, the process comprising reacting a compound of Formula (I-1) with a thiophosphorylating agent in the presence of at least one Catalyst A and at least one Base A in the presence of at least one Solvent A, to form an intermediate compound of Formula (I-1¹) and then quenching with Quenching Reagent A to form a compound of Formula (I): 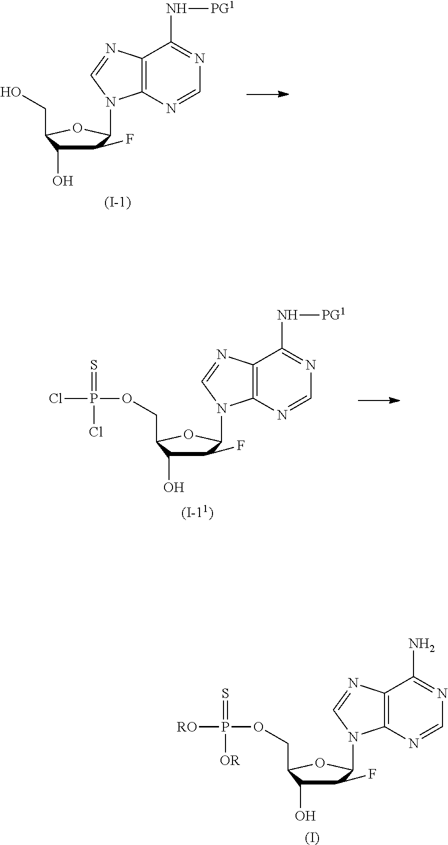
wherein
a) PG¹ is selected from the group consisting of H, isobutyryl, pivaloyl, benzoyl, acetyl, octanoyl, and 2-ethyl-hexanoyl;
b) the thiophosphorylating agent is selected from the group consisting of PSCl₂OK and PSCl₃;
c) the Catalyst A is selected from the group consisting of N-methyl morpholine, N-methyl imidazole, N-methyl-benzimidazole, quinine, 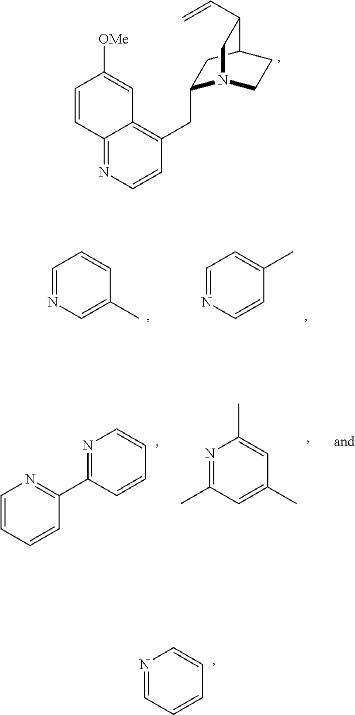
and mixtures thereof;
d) the at least one Base A is selected from the group consisting of 2,6-lutidine, pyridine, 4-picoline, pyridine, 2-picoline, quinoline, 2-F-pyridine, 2,4-lutidine, 2-methyl-pyridine, 2,4,6-trimethylpyridine, 2,3,5-trimethylpyridine, 3-methoxy-pyridine, 4-methyl-pyridine, quinuclidine, Hunig's base, triethylamine, 3-methyl-pyridine, and 2,6-di-tert-butyl-4-methyl pyridine, N-methyl morpholine, and mixtures thereof;
e) the at least one Solvent A is selected from the group consisting of THF, MeCN, acetone, DMPU, HFIP, TFE, glyme, DME, DMAc, propylene carbonate, tetraglyme, trimethyl phosphate, triethyl phosphate, 2-Me-THF, EtOAc, and MIBK, and mixtures thereof; and
f) the at least one Quenching Reagent A selected from the group consisting of water, water in combination with pyridine, or water in combination with one or more additives, where said additives are independently selected from guanidine-HCl, phenol, sodium dodecyl sulfate, thiourea, lithium acetate, magnesium chloride, and urea, and mixtures thereof.

2. The process according to claim 1, wherein the compound of Formula (I) is a compound of Formula (Ia), or a pharmaceutically acceptable salt, hydrate, or solvate thereof:

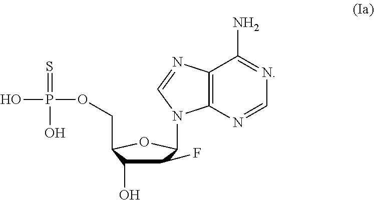

3. The process according to claim 1, wherein the thiophosphorylating agent is PSCl₃.

4. The process according to claim 1, wherein the Catalyst A is selected from the group consisting of 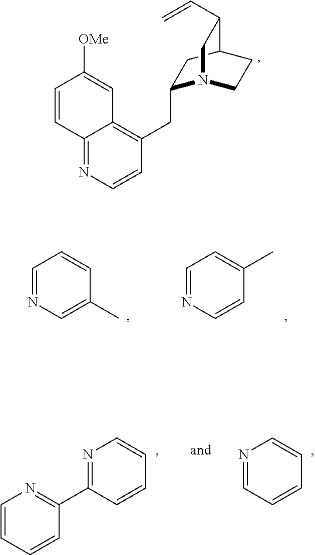
and mixtures thereof.

5. The process according to claim 1, wherein the at least one Base A is selected from the group consisting of 2,6-lutidine, 2,4,6-trimethylpyridine, 2,3,5-trimethylpyridine, 2,4-dimethylpyridine, and pyridine, and mixtures thereof.

6. The process according to claim 1, wherein the at least one Solvent A is selected from the group consisting of tetraglyme, MeCN, trimethyl phosphate, and triethyl phosphate, and mixtures thereof.

7. The process according to claim 1, wherein the at least one Quenching Reagent A is water.

8. The process according to claim 1, wherein the at least one Quenching Reagent A is selected from the group consisting of water in combination with pyridine, or water in combination with one or more additives, where said additives are independently selected from guanidine-HCl, phenol, sodium dodecyl sulfate, thiourea, lithium acetate, magnesium chloride, and urea, and mixtures thereof.

9. The process according to claim 1, further comprising isolating the compound of Formula (I) by crystallization from at least one Solvent B selected from the group of water, methanol, ethanol, isopropanol, and mixtures thereof.

10. The process according to claim 1, further comprising reacting a compound of Formula (I-2²) with at least one Acid A in the presence of at least one Solvent E to provide a compound of Formula (I-1): 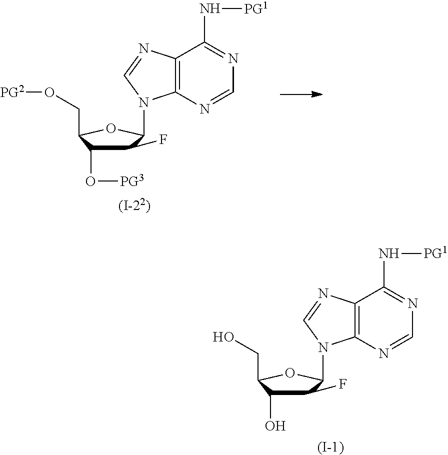
wherein
a) PG¹ is selected from the group consisting of H, isobutyryl, pivaloyl, benzoyl, acetyl, octanoyl, 2-ethyl-hexanoyl;
b) PG² is selected from the group consisting of acyl halides, alkyl halides, and silyl halides; and
c) PG³ is selected from the group consisting of acyl halides, alkyl halides, and silyl halides;
d) the at least one Acid A is selected from the group consisting of TFA, HCl, H₂SO₄, MsOH, TsOH, and mixtures thereof; and
e) the at least one Solvent E is selected from the group consisting of isopropanol, methanol, water, ethyl acetate, and ethanol, and mixtures thereof.

11. The process according to claim 10, further comprising reacting a compound of Formula (I-2¹) with a protected adenine in the presence of at least one Base C, and at least one Solvent D to provide a compound of Formula (I-2²): 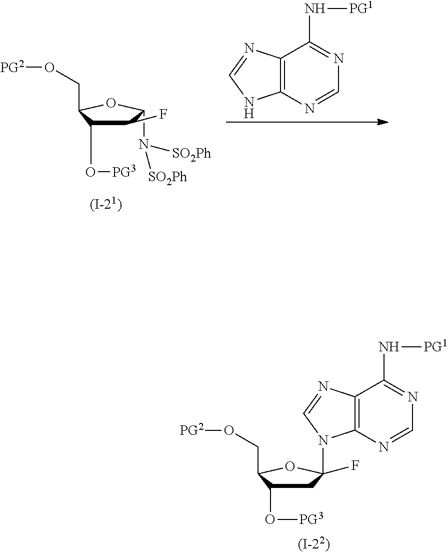
wherein
a) PG¹ is selected from the group consisting of H, isobutyryl, pivaloyl, benzoyl, acetyl, octanoyl, 2-ethyl-hexanoyl;
b) PG² is selected from the group consisting of acyl halides, alkyl halides, and silyl halides; and
c) PG³ is selected from the group consisting of acyl halides, alkyl halides, and silyl halides;
d) the at least one Base C is selected from the group consisting of N,N-diisopropylethylamine, 2,6-lutidine, 2,2,6,6-tetramethylpiperidine, potassium acetate, potassium tert-butoxide, potassium carbonate, tribasic potassium phosphate, N-methylmorpholine, potassium bicarbonate, sodium bicarbonate, sodium carbonate, cesium carbonate, DBU, 2,4,6-collidine, DABCO, N-methylimidazole, 2,6-ditert-butyl-4-methyl pyridine, potassium tert-butoxide, and potassium hexamethyldisilazide, and mixtures thereof; and
e) the at least one Solvent D is selected from the group consisting of toluene, tetrahydrofuran, acetonitrile, dimethylformamide, acetone, dimethylacetamide, cyclopentyl methyl ether, dimethoxyethane, diethylcarbonate, 2-methyl tetrahydrofuran, isopropyl acetate, and ethyl acetate, and mixtures thereof.

12. The process according to claim 11, further comprising reacting a compound of Formula (I-2) with at least one Fluorinating Agent A in the presence of at least one Base B, at least one Silylating Reagent A, and at least one Solvent C to provide a compound of Formula (I-2¹): 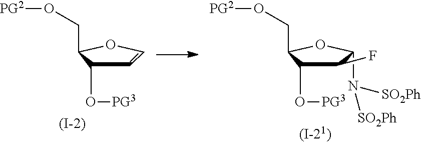
wherein
a) PG¹ is selected from the group consisting of H, isobutyryl, pivaloyl, benzoyl, acetyl, octanoyl, 2-ethyl-hexanoyl;
b) PG² is selected from the group consisting of acyl halides, alkyl halides, and silyl halides;
c) PG³ is selected from the group consisting of acyl halides, alkyl halides, and silyl halides;
d) the at least one Fluorinating Agent A is selected from the group consisting of N-fluorobenzenesulfonimide, 1-chloromethyl-4-fluoro-1,4-diazoniabicyclo[2.2.2]octane bis(tetrafluoroborate), 1-fluoro-4-methyl-1,4-diazoniabicyclo[2.2.2] octanebis(tetrafluoroborate), N-fluoropyridinium triflate, and N-fluoropyridinium tetrafluoroborate, and mixtures thereof;
e) the at least one Base B is selected from the group consisting of pyridine, 2,6-lutidine, triethylamine, N-methylmorpholine, 2,4,6-collidine, potassium carbonate, dibasic potassium phosphate, tribasic potassium phosphate, and sodium bicarbonate, and mixtures thereof;
f) the at least one Silylating Reagent A is selected from the group consisting of 1,3-bis(trimethylsilyl)urea, 3-(trimethylsilyl)-oxazolidin-2-one, hexamethyldisilazane, bistrimethylsilyl acetamide, bistrimethylsilyl trifluoroacetamide, and N-trimethylsilylimidazole, and mixtures thereof; and
g) the at least one Solvent C is selected from the group consisting of ethyl acetate, dioxane, dimethoxyethane, tetrahydrofuran, 2-methyltetrahydrofuran, cyclopentyl methylether, methyl tert-butyl ether, 1,2-dichloroethane, acetonitrile, isopropyl acetate, diethylcarbonate, and toluene, and mixtures thereof.

13. The process according to claim 12, further comprising reacting a compound of Formula (I-3) with PG²-X and PG³-X in the presence of at least one Base D, at least one Silylating Reagent B, at least one Catalyst B, and at least one Solvent F to provide a compound of Formula (I-2): 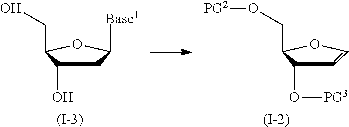
wherein
a) Base¹ is selected from the group consisting of thymine, uracil, cytosine, N-acetylcytosine, guanine, and hypoxanthine;
b) PG²-X is selected from the group consisting of acyl halides, alkyl halides, and silyl halides;
c) PG³-X is selected from the group consisting of acyl halides, alkyl halides, and silyl halides;
d) PG² is selected from the group consisting of acyl halides, alkyl halides, and silyl halides;
e) PG³ is selected from the group consisting of acyl halides, alkyl halides, and silyl halides;
f) the at least one Base D is selected from the group consisting of Hunig's Base, imidazole, pyridine, NMI, 2,6-lutidine, 2,4,6-collidine, DBU, DABCO, tetramethylguanidine, triethylamine, diisopropylethylamine, and mixtures thereof;
g) the at least one Silylating Reagent B is selected from the group consisting of 1,3-bis(trimethylsilyl)urea, 3-(trimethylsilyl)-oxazolidin-2-one, hexamethyldisilazane, bistrimethylsilyl acetamide, bistrimethylsilyl trifluoroacetamide, and N-trimethylsilylimidazole, and mixtures thereof;
h) the at least one Catalyst B is selected from the group consisting of Bronsted acid catalysts, and mixtures thereof; and
i) the at least one Solvent F, which is selected from the group consisting of dichloromethane, dichloroethane, hexane, heptane, cyclohexane, CPME, toluene, trifluorotoluene, hexamethyldisiloxane, hexamethyldisilazane, or a mixture thereof.

14. The process according to claim 13, wherein compound (I-3) is selected from the group consisting of 2′-deoxynucleosides.

15. The process according to claim 14, wherein compound (I-3) is selected from the group consisting of thymidine, 2′-deoxyluridine, 2′-deoxycytidine, 2′-deoxyguanosine, and 2′-deoxyinosine.

16. The process according to claim 14, the at least one Catalyst B is selected from the group consisting of sulfuric acid, methanesulfonic acid, N,N-bistriflimide, 1,2-phenyldisulfonimide, N,N-dibenzenesulfonimide, N,N-bis(4-methoxybenzenesulfonyl)amide, N-(4-chlorobenzenesulfonyl)-N-methanesulfonylamide, N-benzenesulfonyl-benzamide, N,N-bis(methanesulfonyl)amide, saccharin, thiosaccharin, 6-nitrosaccharin, 6-chlorosaccharin, 5-fluorosaccharin, perfluorobenzenesulfonamide, diphenyldithiophosphinic acid, diethyldithiophosphoric acid, N,N-bis(diphenylthiophosphoryl)amide, and N,N-bis(diphenylselenophosphoryl)amide, and mixtures thereof.

17. A process for preparation of a compound of Formula (I), or a pharmaceutically acceptable salt, hydrate, or solvate thereof: 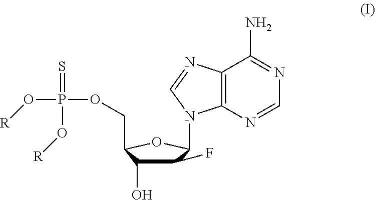
wherein each R is independently selected from the group consisting of H, Na, and K, comprising
i) reacting a compound of Formula (I-3) with PG²-X and PG³-X in the presence of at least one Base D, at least one Silylating Reagent B, at least one Catalyst B, and at least one Solvent F to provide a compound of Formula (I-2): 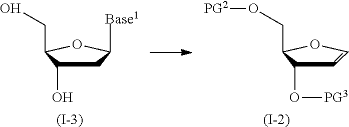
wherein
a) Base¹ is selected from the group consisting of thymine, uracil, cytosine, N-acetylcytosine, guanine, and hypoxanthine;
b) PG²-X is selected from the group consisting of acyl halides, alkyl halides, and silyl halides;
c) PG³-X is selected from the group consisting of acyl halides, alkyl halides, and silyl halides;
d) PG² is selected from the group consisting of acyl halides, alkyl halides, and silyl halides;
e) PG³ is selected from the group consisting of acyl halides, alkyl halides, and silyl halides;
f) the at least one Base D is selected from the group consisting of amines, and mixtures thereof;
g) the at least one Silylating Reagent B is selected from the group consisting of 1,3-bis(trimethylsilyl)urea, 3-(trimethylsilyl)-oxazolidin-2-one, hexamethyldisilazane, bistrimethylsilyl acetamide, bistrimethylsilyl trifluoroacetamide, and N-trimethylsilylimidazole, and mixtures thereof;
h) the at least one Catalyst B is selected from the group consisting of Bronsted acid catalysts, and mixtures thereof;
i) the at least one Solvent F is selected from the group consisting of hydrocarbons, halocarbons, ethers, and silanes, and mixtures thereof; and
j) optionally purifying the compound of Formula (I-2) by adding the reaction mixture to an alcohol or mixture of alcohols to induce a selective alcoholysis and precipitation of by-products;
ii) reacting a compound of Formula (I-2) with at least one Fluorinating Agent A in the presence of at least one Base B, at least one Silylating Reagent A, and at least one Solvent C to provide a compound of Formula (I-2¹): 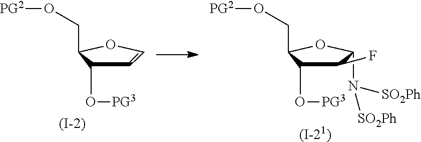
wherein
a) the at least one Fluorinating Agent A is selected from the group consisting of N-fluorobenzenesulfonimide, 1-chloromethyl-4-fluoro-1,4-diazoniabicyclo[2.2.2]octane bis(tetrafluoroborate), 1-fluoro-4-methyl-1,4-diazoniabicyclo[2.2.2] octanebis(tetrafluoroborate), N-fluoropyridinium triflate, and N-fluoropyridinium tetrafluoroborate, and mixtures thereof;
b) the at least one Base B is selected from the group consisting of pyridine, 2,6-lutidine, triethylamine, N-methylmorpholine, 2,4,6-collidine, potassium carbonate, dibasic potassium phosphate, tribasic potassium phosphate, and sodium bicarbonate, and mixtures thereof;
c) the at least one Silylating Reagent A is selected from the group consisting of 1,3-bis(trimethylsilyl)urea, 3-(trimethylsilyl)-oxazolidin-2-one, hexamethyldisilazane, bistrimethylsilyl acetamide, bistrimethylsilyl trifluoroacetamide, and N-trimethylsilylimidazole, and mixtures thereof; and
d) the at least one Solvent C is selected from the group consisting of ethyl acetate, dioxane, dimethoxyethane, tetrahydrofuran, 2-methyltetrahydrofuran, cyclopentyl methylether, methyl tert-butyl ether, 1,2-dichloroethane, acetonitrile, isopropyl acetate, diethylcarbonate, and toluene, and mixtures thereof;
iii) reacting a compound of Formula (I-2¹) with a protected adenine in the presence of at least one Base C, and at least one Solvent D to provide a compound of Formula (I-2²): 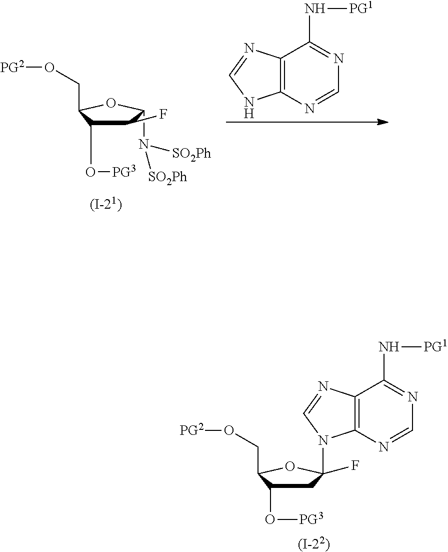
wherein
a) PG¹ is selected from the group consisting of H, isobutyryl, pivaloyl, benzoyl, acetyl, octanoyl, 2-ethyl-hexanoyl;
b) the at least one Base C is selected from the group consisting of N,N-diisopropylethylamine, 2,6-lutidine, 2,2,6,6-tetramethylpiperidine, potassium acetate, potassium tert-butoxide, potassium carbonate, tribasic potassium phosphate, N-methylmorpholine, potassium bicarbonate, sodium bicarbonate, sodium carbonate, cesium carbonate, DBU, 2,4,6-collidine, DABCO, N-methylimidazole, 2,6-ditert-butyl-4-methyl pyridine, potassium tert-butoxide, and potassium hexamethyldisilazide, and mixtures thereof; and
c) the at least one Solvent D is selected from the group consisting of toluene, tetrahydrofuran, acetonitrile, dimethylformamide, acetone, dimethylacetamide, cyclopentyl methyl ether, dimethoxyethane, diethylcarbonate, 2-methyl tetrahydrofuran, isopropyl acetate, and ethyl acetate, and mixtures thereof;
iv) reacting a compound of Formula (I-2¹) with at least one Acid A in the presence of at least one Solvent E to provide a compound of Formula (I-1): 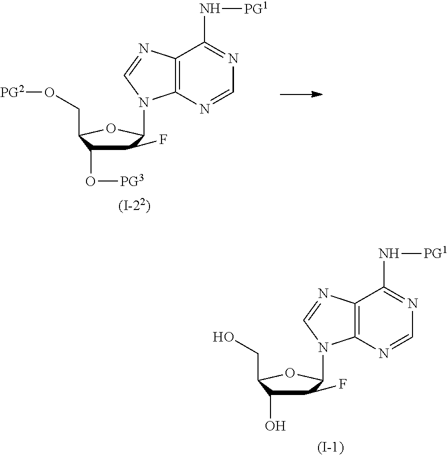
wherein
a) the at least one Acid A is selected from the group consisting of TFA, HCl, H₂SO₄, MsOH, TsOH, and mixtures thereof; and
b) the at least one Solvent E is selected from the group consisting of isopropanol, methanol, water, ethyl acetate, and ethanol, and mixtures thereof; and
v) reacting the compound of Formula (I-1) with a thiophosphorylating agent in the presence of at least one Catalyst A and at least one Base A in the presence of at least one Solvent A, to form an intermediate compound of Formula (I-1¹) and then quenching with Quenching Reagent A to form a compound of Formula (I): 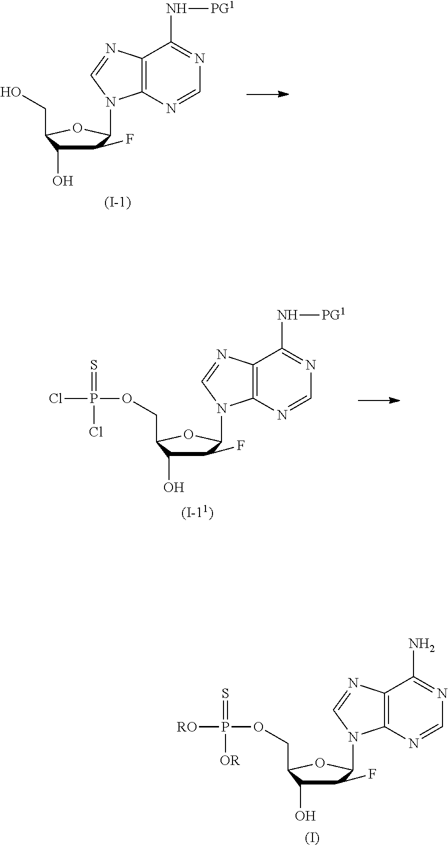
wherein
a) R is H;
b) PG¹ is selected from the group consisting of H, isobutyryl, pivaloyl, benzoyl, acetyl, octanoyl, 2-ethyl-hexanoyl;
c) the thiophosphorylating agent is selected from the group consisting of PSCl₂OK and PSCl₃;
d) the Catalyst A is selected from the group consisting of N-methyl morpholine, N-methyl imidazole, N-methyl-benzimidazole, quinine, 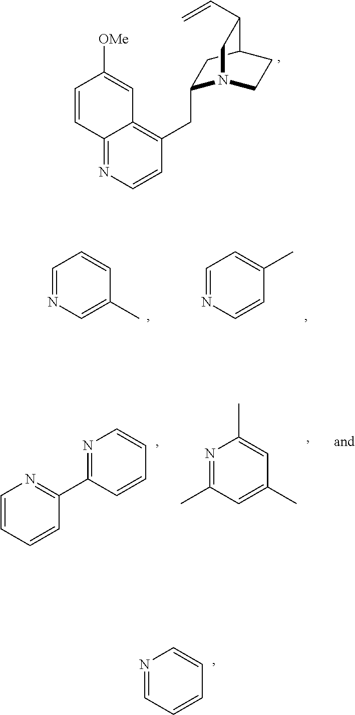
and mixtures thereof;
e) the at least one Base A is selected from the group consisting of 2,6-lutidine, pyridine, 4-picoline, pyridine, 2-picoline, quinoline, 2-F-pyridine, 2,4-lutidine, 2-methyl-pyridine, 2,4,6-trimethylpyridine, 2,3,5-trimethylpyridine, 3-methoxy-pyridine, 4-methyl-pyridine, quinuclidine, Hunig's base, triethylamine, 3-methyl-pyridine, and 2,6-di-tert-butyl-4-methyl pyridine, N-methyl morpholine, and mixtures thereof;
f) the at least one Solvent A is selected from the group consisting of THF, MeCN, acetone, DMPU, HFIP, TFE, glyme, DME, DMAc, propylene carbonate, tetraglyme, trimethyl phosphate, triethyl phosphate, 2-Me-THF, EtOAc, and MIBK, and mixtures thereof; and
g) the at least one Quenching Reagent A is selected from the group consisting of water, water in combination with pyridine, or water in combination with one or more additives, where said additives are independently selected from guanidine-HCl, phenol, sodium dodecyl sulfate, thiourea, lithium acetate, magnesium chloride, and urea, and mixtures thereof.

18. The process according to claim 17, further comprising isolating the compound of Formula (I) by crystallization from at least one Solvent B selected from the group of water, methanol, ethanol, isopropanol and mixtures thereof.

19. The process according to claim 17, further comprising forming a sodium or potassium salt of the compound of Formula (I).

20. A process for preparation of a compound of Formula (Ia), or a pharmaceutically acceptable salt, hydrate, or solvate thereof: 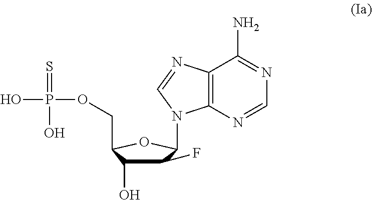
comprising
i) reacting thymidine with trimethylsilyl chloride in the presence of imidazole, bistrimethylsilyl acetamide, at least one Catalyst B is selected from the group consisting of N,N-dibenzenesulfonimide, N,N-bis(diphenylthiophosphoryl)amide, and N,N-bis(diphenylselenophosphoryl) amide, and mixtures thereof, and at least one Solvent F, is selected from the group consisting of dichloromethane, dichloroethane, hexane, heptane, cyclohexane, CPME, toluene, trifluorotoluene, hexamethyldisiloxane, and hexamethyldisilazane, and mixtures thereof: 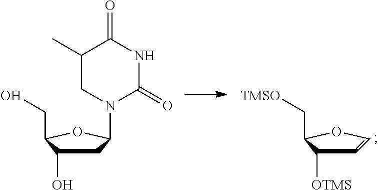
ii) reacting the product of step i) successively
a) with N-fluorobenzenesulfonimide in the presence of 2,6-lutidine, bistrimethylsilyl trifluoroacetamide, and toluene,
b) with pivaloyl-protected adenine in the presence of 2,6-lutidine and ethyl acetate,
c) with TFA in the presence of ethanol:

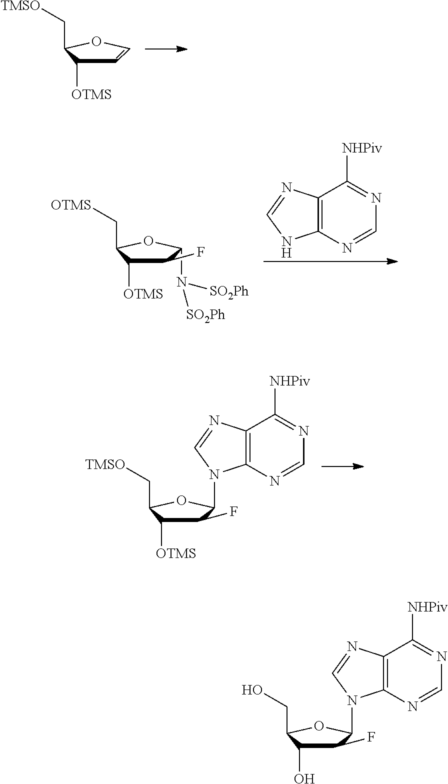

21. The process according to claim 20, further comprising forming a sodium or potassium salt of the compound of Formula (Ia).

22. A compound selected from the group consisting of: [chemical structure]
